(19) United States
(12) Patent Application Publication
(10) Pub. No.: US 2012/0195935 A1
(43) Pub. Date: Aug. 2, 2012

(54) MICROBUBBLE ASSISTED VIRAL DELIVERY

(75) Inventors: Paul B. Fisher, Richmond, VA (US); Devanand Sarkar, Richmond, VA (US); Rupesh Dash, Richmond, VA (US); Belal Mohammed Azab, Richmond, VA (US); Xiang-Yang Wang, Richmond, VA (US); Pier Paolo Claudio, Huntington, WV (US)

(73) Assignee: VIRGINIA COMMONWEALTH UNIVERSITY, Richmond, VA (US)

(21) Appl. No.: 13/386,165

(22) PCT Filed: Jul. 27, 2010
(86) PCT No.: PCT/US10/43403

§ 371 Date: Apr. 13, 2012

Related U.S. Application Data
(60) Provisional application No. 61/228,721, filed on Jul. 27, 2009.

(51) Int. Cl. A61K 9/00; A61P 35/00; A61M 37/00; A61K 35/76

(52) U.S. Cl. 424/400; 424/93.6; 604/22

(57) ABSTRACT

Microbubble-assisted delivery of viruses is disclosed. In particular, methods for targeting a virus to cancer cells in an immunocompetent animal by administering a selectively replicating virus to the immunocompetent animal and disrupting the microbubbles in a location of the animal comprising cancer cells are provided. The virus is encompassed in a suspension of microbubbles, and the surface of the suspension does not include any virus. A suspension of microbubbles comprising a selectively replicating virus that is encompassed in a suspension of microbubbles, which does not include any virus on the surface of the suspension, is also provided.

### CROSS-REFERENCES TO RELATED APPLICATIONS

The application claims benefit of priority to U.S. Provisional Application No. 61/228,721, filed Jul. 27, 2009, the disclosure of which is incorporated by reference herein in its entirety.

### FEDERALLY SPONSORED RESEARCH AND DEVELOPMENT

The subject matter of this application was made with Government support under Grant Nos. CA138510, CA104177, R01 CA35675 and R01 CA097318 awarded by the National Institutes of Health (NIH). The Government has certain rights in the invention(s) provided herein.

### BACKGROUND OF THE INVENTION

Prostate cancer is the most common cancer and the second leading cause of cancer related deaths in men in the United States (Damber, J E, and Aus, G (2008). Prostate cancer. Lancet 371: 1710-1721). At present, no effective therapy is available for metastatic prostate cancer (PC) (Moon, C, Park, J C, Chae, Y I, Yun, J H, and Kim, S (2008). Current status of experimental therapeutics for prostate cancer. Cancer Lett 266: 116-134). Advanced PC is refractory to conventional anticancer treatments because of frequent overexpression of antiapoptotic proteins Bcl-2 and/or Bcl-XL (Shi, X B, Gumerlock, P H, and deVere White, R W (1996). Molecular biology of prostate cancer. World J Uro 114: 318-328; Lebedeva, I V, et al. (2003). Bcl-2 and Bcl-x(L) differentially protect human prostate cancer cells from induction of apoptosis by melanoma differentiation associated gene-7, mda-7/IL-24. Oncogene 22: 8758-8773). The Melanoma differentiation associated gene7/interleukin-24 (mda-7/IL-24), is a secreted cytokine having broad-spectrum, cancer-selective, apoptosis-inducing properties that profoundly inhibits prostate cancer cell growth (Lebedeva, I V, et al. (2003). Melanoma differentiation associated gene-7, mda7/interleukin-24, induces apoptosis in prostate cancer cells by promoting mitochondrial dysfunction and inducing reactive oxygen species. Cancer Res 63: 8138-8144). Adenovirus (Ad)-mediated delivery of mda-7/IL-24 (Ad.mda-7) has shown dramatic anti-tumor effects in animal models and in clinical trials (Fisher, P B (2005) Is mda-7/IL-24 a “magic bullet” for cancer? Cancer Res 65: 10128-10138; Fisher, P B, et al. (2003) mda-7/IL-24, a novel cancer selective apoptosis inducing cytokine gene: from the laboratory into the clinic. Cancer Biol Ther 2: S23-37; Lebedeva, I V, et al. (2007). mda-7/IL-24, novel anticancer cytokine focus on bystander antitumor, radiosensitization and antiangiogenic properties and overview of the phase I clinical experience (Review). Int J Oncol 31: 985-1007; Lebedeva, I V, et al. (2005) mda-7/IL-24: exploiting cancer's Achilles' heel. Mol Ther 11: 4-18). However, forced overexpression of Bcl-2 or Bcl-XL renders prostate cancer cells resistant to Ad.mda-7 (Lebedeva, I V, et al. (2003). Bcl-2 and Bcl-x(L) differentially protect human prostate cancer cells from induction of apoptosis by melanoma differentiation associated gene-7, mda-7/IL-24. Oncogene 22: 8758-8773). In contrast, a conditionally replication-competent adenovirus (CRCA) (a cancer terminator virus—CTV), which expresses mda-7/IL-24 (Ad.PEG-E1 A-mda-7) can abrogate acquired resistance of prostate cancer cells mediated through Bcl-2 and/or BclXL overexpression causing growth arrest and apoptosis and selectively replicating in prostate cancer xenografted cells in athymic nude mice. Moreover, the CTV completely eradicates not only primary tumors but also distant tumors following repeated intratumoral injections into the primary tumor site (Sarkar, D, Dent, P, Curiel, D T, and Fisher, P B (2008). Acquired and innate resistance to the cancer-specific apoptosis-inducing cytokine, mda-7/IL-24: not insurmountable therapeutic problems. Cancer Bioi Ther 7: 109-112; Sarkar, D, et al. (2007). Eradication of therapy-resistant human prostate tumors using a cancer terminator virus. Cancer Res 67: 5434-544).

### BRIEF SUMMARY OF THE INVENTION

The invention provides methods of targeting a virus to cancer cells in an immunocompetent animal. In some embodiments, the method comprises administering a selectively replicating virus to the immunocompetent animal, wherein the virus is encompassed in a suspension of microbubbles, wherein the surface of the suspension does not include any virus; and disrupting the microbubbles administered to the animal in a location of the animal comprising cancer cells, wherein the virus selectively replicates in the cancer cell.

In some embodiments, the animal is a human.

In some embodiments, the cancer cell is killed as a result of replication or gene expression of the virus in the cancer cell.

In some embodiments, the virus further expresses a chaperone protein, wherein the chaperone protein presents killed cancer cell antigens to the animal immune system. In some embodiments, the chaperone protein is Grp170.

In some embodiments, the virus comprises a cancer-specific promoter operably linked to at least one viral gene necessary for viral replication, thereby rendering the virus selectively replicating. In some embodiments, the promoter is a human PEG-3 promoter.

In some embodiments, the virus further expresses a heterologous polynucleotide, wherein the heterologous polynucleotide encodes mda-7. In some embodiments, expression of the heterologous polynucleotide is under the control of a human PEG-3 promoter.

In some embodiments, the virus is an adenovirus.

In some embodiments, the virus and/or suspension of microbubbles have altered tropism.

The invention also provides for suspensions of microbubbles, the suspension including a selectively replicating virus wherein the virus is encompassed in a suspension of microbubbles and the surface of the suspension does not include any virus.

In some embodiments, the virus selectively replicates in cancer cells.

In some embodiments, a cancer cell is killed as a result of replication or gene expression of the virus in the cancer cell, wherein the virus further expresses a chaperone protein, wherein the chaperone protein is capable of presenting killed cancer cell antigens to the animal immune system.

In some embodiments, the chaperone protein is Grp170.

In some embodiments, the virus comprises a cancer-specific promoter operably linked to at least one viral gene necessary of viral replication, thereby rendering the virus selectively replicating. In some embodiments, the promoter is a human PEG-3 promoter.

In some embodiments, the virus further expressed a heterologous polynucleotide, wherein the heterologous polynucleotide encodes mda-7.

In some embodiments, expression of the heterologous polynucleotide is under the control of a human PEG-3 promoter.

In some embodiments, the virus is an adenovirus.

In some embodiments, the virus and/or suspension of microbubbles have altered tropism.

The invention also provides pharmaceutical compositions comprising the suspension as described above.

Additional features, advantages, and embodiments of the invention may be set forth or apparent from consideration of the following detailed description, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced.

FIG. 1 illustrates A) Schematic representation of the microbubble delivery of Ad-GFP complexes and US release in a tumor target site of the mouse. B) Western Blot Analysis of Ad-GFP microbubble-transduced DU-145 tumor xenografts. Immunoblot showing the expression levels of GFP in DU-145 cells following ultrasound targeted microbubble-Ad transduction of GFP at 96 hr. Only the tumor on the right flank was sonoporated for 10 min resulting in the delivery and expression of GFP. The left tumor, heart, lung, liver, and kidney were negative for GFP expression. Purified GST-GFP was used as a positive control. Protein gel loading was normalized using β-actin as a control.

FIG. 2 illustrates ultrasound imaging and US contrast enhancement of in vivo transduced DU-145 tumor xenografts. Panel A. B-Mode US imaging of a tumor before MB contrast agent injection. Panel B. B-Mode US imaging of the same tumor depicted in panel A following injection of microbubbles/Ad-GFP complexes. MBs cavitation within the targeted tumor dramatically enhances the tumor image within the US field of view.

FIG. 3 illustrates growth curves and Western blot analysis of large DU-145 and DU-Bcl-xL tumor xenografts treated with microbubble encapsulated Ad-GFP, Ad.mda-7 or CTV (Ad.PEG-E1A-mda-7) and treated with US in the right tumor. Subcutaneous tumor xenografts from DU-145 and DU-Bcl-xL were established in athymic nude mice in both right and left flanks and only tumors on the right side were sonoporated following tail vein injection of the indicated microbubble/Ad complexes during a course of 4 wks. Tumor treatments were initiated when tumors reached a size of 250-350 mm³. Arrows point at tumors and asterisks point at treatment times. A) Measurement of GFP-treated DU-145 tumor volumes. The data represent mean±s.d. with at least 5 mice in each group. B) Measurement of Ad.mda-7-treated DU-145 tumor volumes. The data represent mean±s.d. with at least 7 mice in each group. C) Measurement of CTV-treated DU-145 tumor volumes. The data represent mean±s.d. with at least 7 mice in each group. D) Western blot analysis of protein extracts from representative DU-145 tumor samples treated with Ad.mda-7 or CTV. The immunoblot was reacted with anti-MDA-7/IL-24. Arrowheads point at the various glycolsylated forms of MDA-7/IL-24. Protein gel loading was normalized using anti-GAPDH as a control. E) Measurement of GFP-treated DU-Bcl-xL tumor volumes. The data represent mean±s.d. with at least 5 mice in each group. F) Measurement of Ad.mda-7-treated DU-Bcl-xL tumor volumes. The data represent mean±s.d. with at least 7 mice in each group. G) Measurement of CTV-treated DU-Bcl-xL tumor volumes. The data represent mean±s.d. with at least 7 mice in each group. H) Western blot analysis of protein extracts from representative DU-Bcl-xL tumor samples treated with Ad.mda-7 or CTV. The immunoblot was reacted with anti-MDA-7/IL-24. Arrowheads point at the various glycolsylated forms of MDA-7/IL-24. Protein gel loading was normalized using anti-GAPDH as a control.

FIG. 4 illustrates growth curves of DU-145 tumor xenografts treated with microbubble/ultrasound guided Ad-GFP, Ad.mda-7, or CTV. Subcutaneous tumor xenografts from DU-145 cells were established in athymic nude mice in both right and left flanks and only tumors on the right side were sonoporated following tail vein injection of the indicated microbubble/Ad complexes during a course of 4 weeks. Tumor treatments were initiated when tumors reached a size of 25-35 mm³. A, C, and E) Photographs of animals representative of each group. Black arrows indicate tumors. B) Measurement of GFP-treated DU-145 tumor volumes. The data represent mean±s.d. with at least 6 mice in each group. D) Measurement of Ad.mda-7-treated DU-145 tumor volumes. The data represent mean±s.d. with at least 9 mice in each group. F) Measurement of CTV-treated DU-145 tumor volumes. The data represent mean±s.d. with at least 9 mice in each group. Asterisks indicate treatment times.

FIG. 5 illustrates Colorimetric TUNEL assay of DU-145 tumor xenografts treated with microbubble/ultrasound guided Ad-GFP, Ad.mda-7, or CTV. Subcutaneous tumor xenografts from DU-145 cells were established in athymic nude mice in both right and left flanks and only tumors on the right side were sonoporated following tail vein injection of the indicated microbubble/Ad complexes during a course of 4 weeks. Tumors were removed, fixed, sectioned and stained to determine levels of double-stranded DNA breaks (TUNEL). Microscopy for TUNEL sections was under visible light at 40× magnification (a representative of 3-separate tumors). A-B) TUNEL staining of left and right side DU-145 tumors following systemic injection of Ad-GFP-microbubble plus US treatment of the tumor on the right side. C-D) TUNEL staining of left and right side DU-145 tumors following systemic injection of Ad.mda-7-microbubble plus US treatment of the tumor on the right side. E-F) TUNEL staining of left and right side DU-145 tumors following systemic injection of CTV-microbubble plus US treatment of the tumor on the right side.

FIG. 6 illustrates growth curves of therapy resistant DU-Bcl-xL tumor xenografts treated with microbubble/ultrasound guided Ad-GFP, Ad. mda-7, or CTV. Subcutaneous tumor xenografts from DU-Bcl-xL cells were established in athymic nude mice in both right and left flanks and only tumors on the right side were sonoporated following tail vein injection of the indicated microbubble/Ad complexes during a course of 4 weeks. Arrows point at tumors and asterisks point at treatment times. A, C, and E) Photographs of animals representative of each group. Black arrows point at tumors. B) Measurement of Ad-GFP-microbubble-treated DU-Bcl-xL tumor volumes. The data represent mean±s.d. with a minimum of 6 mice in each group. D) Measurement of Ad.mda-7-microbubble-treated DU-Bcl-xL tumor volumes. The data represent mean±s.d. with a minimum of 9 mice in each group. F) Measurement of CTV-microbubble-treated DU-Bcl-xL tumor volumes. The data represent mean±s.d. with a minimum of 9 mice in each group.

FIG. 7 illustrates colorimetric TUNEL assay of therapy resistant DU-Bcl-xL tumor xenografts treated with microbubble/US guided Ad-GFP, Ad.mda-7, or CTV. Subcutaneous tumor xenografts from DU-Bcl-xL cells were established in athymic nude mice in both right and left flanks, and only tumors on the right side were sonoporated following tail vein injection of the indicated microbubble/Adenoviral complexes during a course of 4 weeks. Tumors were removed fixed, sectioned and stained to determine levels of double-stranded DNA breaks (TUNEL). Microscopy for TUNEL sections was under visible light at ×40 magnification (a representative of 3 separate tumors). A-B) TUNEL staining of left and right side DU-Bcl-xL tumors following Ad-GFP-microbubble treatment. C-D) TUNEL staining of left and right side DU-Bcl-xL tumors following Ad.mda-7-microbubble treatment. E-F) TUNEL staining of left and right side DU-Bcl-xL tumors following CTV-microbubble treatment.

FIG. 8 illustrates immunohistochemical analysis of DU-145 and therapy resistant DU-Bcl-xL tumor xenografts treated with microbubble/US guided Ad-GFP, Ad.mda-7, or CTV. Subcutaneous tumor xenografts from DU-145 or DU-Bcl-xL cells were established in athymic nude mice in both right and left flanks, and only tumors on the right side were sonoporated following tail vein injection of the indicated microbubble/Adenoviral complexes during a course of 4 weeks. Tumors were removed fixed, sectioned and immunostained to determine levels of E1A expression. A) E1A immunohistochemical staining of control normal tissue from a DU-145 mouse treated with CTV microbubbles. B) E1A immunohistochemical staining of control tumor tissues from a DU-145 mouse treated with Ad-GFP microbubbles and US. C-D) E1A immunohistochemical staining of left and right side DU-145 tumors following CTV-microbubble treatment and US treatment of the tumor on the right side. E) E1A immunohistochemical staining of control normal tissues from a DU-Bcl-xL mouse treated with CTV microbubbles. F) E1A immunohistochemical staining of control tumor tissues from a DU-Bcl-xL mouse treated with Ad-GFP microbubbles and US. G-H) E1A immunohistochemical staining of left and right side DU-Bcl-xL tumors following CTV-microbubble treatment of the tumor on the right side.

FIG. 9 illustrates B-Mode ultrasound imaging of DU-145 tumor xenografts treated with microbubble/ultrasound guided Ad.mda-7 and therapy resistant DU-Bcl-xL tumor xenografts treated with microbubble/ultrasound guided CTV. Subcutaneous tumor xenografts from DU-145 and DU-Bcl-xL cells were established in athymic nude mice in both right and left flanks and only tumors on the right side were sonoporated following tail vein injection of the indicated microbubble/Adenoviral complexes during a course of 4 weeks. Tumor volumes were determined by measuring twice a week the tumors with either a caliper or by ultrasound measurements of the tumor axes. A) Ultrasound image and measurement of a DU-145 tumor before treatment with Ad.mda-7 microbubble complexes and US. B) Ultrasound image and measurement of the same DU-145 tumor 2 weeks following treatments with Ad.mda-7 microbubble complexes and US. C) Ultrasound image and measurement of the same DU-145 tumor 4 weeks following treatments with Ad.mda-7 microbubble complexes and US. D) Ultrasound image and measurement of a DU-Bcl-xL tumor before treatment with CTV microbubble complexes and US. E) Ultrasound image and measurement of the same DU-Bcl-xL tumor 2 weeks following treatments with the CTV microbubble complexes and US. F) Ultrasound image and measurement of the same DU-Bcl-xL tumor 4 weeks following treatments with CTV microbubble complexes and US. Complete eradication of the DU-Bcl-xL tumor occurs 4 weeks after initiating the therapeutic treatment protocol.

FIG. 10 illustrates growth curves of control DU-145 tumor xenografts injected i.v. using unprotected Ad-GFP, Ad.mda-7 or CTV (Ad.PEG-E1A-mda-7) and treated or not with US. Subcutaneous tumor xenografts from DU-145 were established in athymic nude mice in both right and left flanks and only tumors on the right side were sonoporated following tail vein injection of the indicated Ads during a course of 4 wks. Tumor treatments were initiated when tumors reached a size of 150-200 mm³. Asterisks point at treatment times. A) Measurement of CTV-injected and sonoporated DU-145 tumor volumes. The data represent mean±s.d. with at least 5 mice in each group. B) Measurement of CTV-injected, but not sonoporated DU-145 tumor volumes. The data represent mean±s.d. with at least 5 mice in each group. C) Measurement of Ad.mda-7-injected and sonoporated DU-145 tumor volumes. The data represent mean±s.d. with at least 5 mice in each group. D) Measurement of Ad.mda-7-injected, but not sonoporated DU-145 tumor volumes. The data represent mean±s.d. with at least 5 mice in each group. E) Measurement of Ad.GFP-injected and sonoporated DU-145 tumor volumes. The data represent mean±s.d. with at least 5 mice in each group. D) Measurement of Ad.GFP-injected, but not sonoporated DU-145 tumor volumes. The data represent mean±s.d. with at least 5 mice in each group.

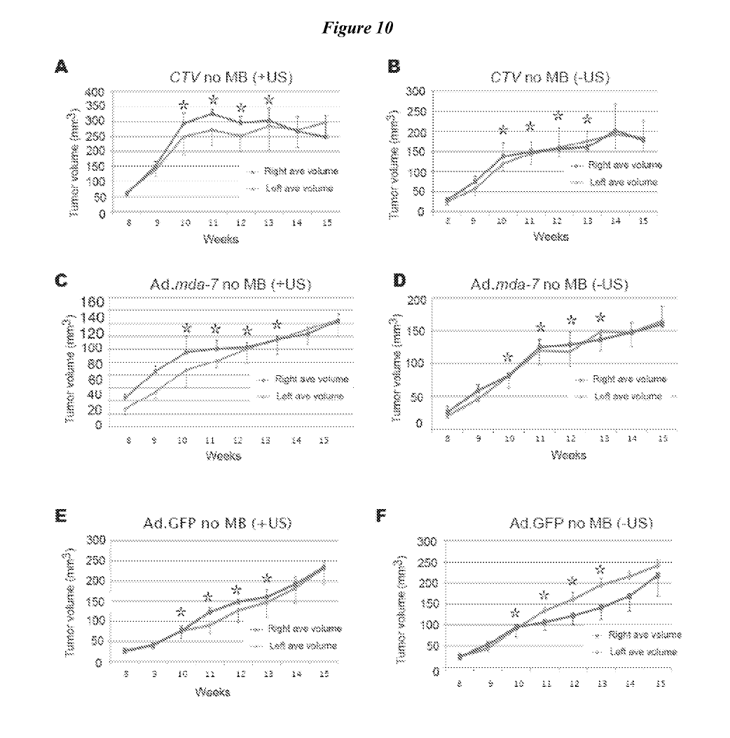

FIG. 11 illustrates growth curves of control DU-Bcl-xL tumor xenografts injected i.v. using unprotected Ad-GFP, Ad.mda-7 or CTV (Ad.PEG-E1A-mda-7) and treated or not with US. Subcutaneous tumor xenografts from DU-Bcl-xL were established in athymic nude mice in both right and left flanks and only tumors on the right side were sonoporated following tail vein injection of the indicated Ads during a course of 4 wks. Tumor treatments were initiated when tumors reached a size of 150-200 mm³. Asterisks point at treatment times. A) Measurement of CTV-injected and sonoporated DU-Bcl-xL tumor volumes. The data represent mean±s.d. with at least 5 mice in each group. B) Measurement of CTV-injected, but not sonoporated DU-Bcl-xL tumor volumes. The data represent mean±s.d. with at least 5 mice in each group. C) Measurement of Ad.mda-7-injected and sonoporated DU-Bcl-xL tumor volumes. The data represent mean±s.d. with at least 5 mice in each group. D) Measurement of Ad.mda-7-injected, but not sonoporated DU-Bcl-xL tumor volumes. The data represent mean±s.d. with at least 5 mice in each group. E) Measurement of Ad.GFP-injected and sonoporated DU-Bcl-xL tumor volumes. The data represent mean±s.d. with at least 5 mice in each group. D) Measurement of Ad.GFP-injected, but not sonoporated DU-Bcl-xL tumor volumes. The data represent mean±s.d. with at least 5 mice in each group.

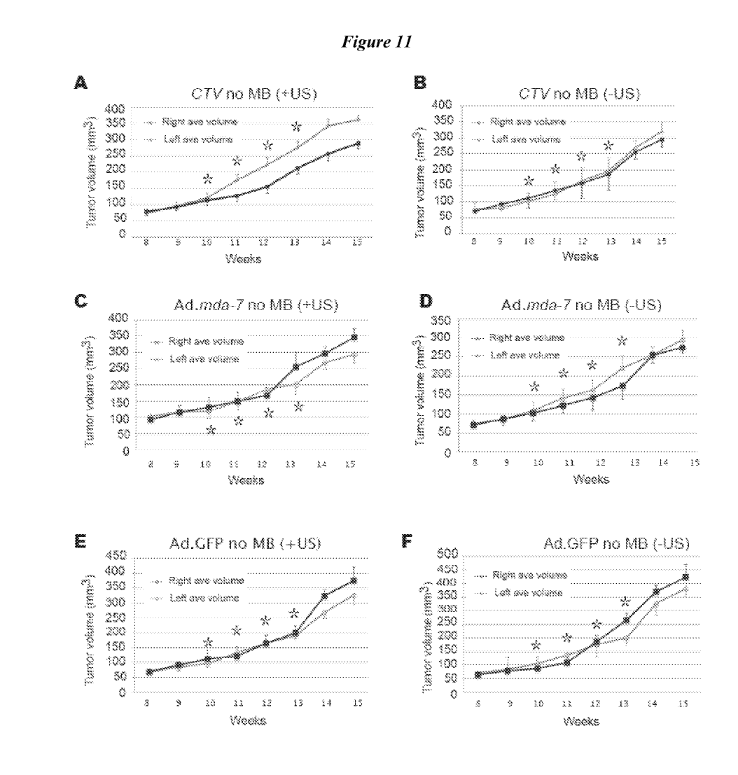

FIG. 12 illustrates adenovirus-mediated expression of secretable grp170 in TRAMP-C2 tumor cell. A. Schematic representation of adenovirus vector encoding a secretable form of grp170 (Ad.sgrp170). The COOH-terminal KNDEL signal was deleted from mouse grp170 cDNA to produce the secreted form of grp170. The His-tagged sgrp170 gene under the control of a constitutively active cytomegalovirus promoter/enhancer (CMV) was inserted into the replication incompetent adenoviral vector, in which the E1/E3 sequences have been deleted. Inverted terminal repeats (ITR), which flank the E1/E3 deleted genome, are necessary for the replication of adenoviral DNA. The TRAMP-C2 cells were infected with or without Ad.sgrp170 at different MOIs. Supernatants were collected from the infected cells at different time points and analyzed for the expression of sgrp170 using antibodies against grp170 (B) or His-tag (C).

FIG. 13 illustrates adenovirus-mediated mda-7 inhibits TRAMP-C2 tumor cell growth by inducing apoptosis. A. Ad.mda-7 infection suppresses proliferation of C2 tumor cell in vitro. C2 cells were infected with Ad.sgrp170, Ad.mda-7 at different MOIs or left untreated. Protein lysates (50 μg) were run on 12% SDS-PAGE and stained with anti-mda-7/IL-24 monoclonal antibody (1:2,000) (top). C2 cells were infected with Ad.GFP, Ad.mda-7 at a MOI of 300 or left untreated. Cell proliferation was analyzed using MTT assay (bottom). B. Ad.mda-7 infection induces C2 tumor cell apoptosis. TRAMP-C2 tumor cells were infected with Ad.GFP, Ad.mda-7 or left untreated. Cells were collected at 72 h after treatment and stained with FITC-labeled Annexin-V. The percentage of Annexin-V positive cells was analyzed by flow cytometry (*p<0.01, Ad.mda-7 versus Ad.GFP or untreated control). C. Ad.mda-7 infection promotes PARP cleavage in C2 tumor cells. C2 cells were infected with or without Ad.GFP or Ad.mda-7. Cells were examined at different time points for cleavage of PARP by immunoblotting. β-actin was used as the internal loading control. D. Ad.mda-7 induces apoptosis in tumor cell but not in normal cells. TRAMP-C2 prostate tumor cells or DC1.2 dendritic cells were infected with Ad.mda-7. Cells were stained with FITC-labeled Annexin-V 48 h later and examined using FACS (dot line—control; solid line—Ad.mda-7 treated cells).

FIG. 14 illustrates intratumoral administration of adenovirus encoding mda-7/IL-24 and secretable grp170 induces a systemic antitumor response. A. Treatment scheme for the combined therapies targeting both tumor and immune compartments. B. Ad.sgrp170 promotes eradication of local C2 tumors by Ad.mda-7 in immunocompetent mice. Male C57BL/6 mice are injected s.c. with TRAMP-C2 tumor cells (n=10, 2×10⁶ cells per mouse). Six days later, mice (n=10) received replication-defective Ad.mda-7, Ad.sgrp170 or Ad.mda-7 plus Ad.sgrp170 i.t. every two days for a total of 4 doses (5×10⁸ pfu per injection). Mice receiving Ad.GFP served as controls. Data are representative of three experiments (*p<0.02, Ad.mda-7 or Ad.sgrp170 versus Ad.GFP on day 42; **p<0.01, Ad.mda-7 plus Ad.sgrp170 versus Ad.mda-7 or Ad.sgrp170). C. Inhibition of established distant tumors by the combined in situ tumor therapies. C2 tumor cells (1.5×10⁶ cells) were inoculated s.c. into the right and left flanks of mice at the same time. Only tumors in the left flank were treated as described above. Growth of the tumors in the contralateral side was followed (*p>0.05, Ad.mda-7 or Ad.sgrp170 versus PBS; **p<0.01, Ad.mda-7 plus Ad.sgrp170 versus Ad.mda-7 or Ad.sgrp170). D. The combined therapies prior to surgery prevent the growth of secondary tumor. C2 tumor cells were inoculated s.c. into the right flank of mice. When tumor volume reached the size of 5 mm in diameters, mice were treated with Ad.GFP, Ad.mda-7, Ad.sgrp170 or Ad.mda-7 plus Ad.sgrp170. Tumors were removed by surgery one week after the last treatment. Mice were rechallenged with C2 tumor cells in the left flank 10 d after surgical removal of the treated primary tumor (*p>0.05, Ad.mda-7 or Ad.sgrp170 versus control; **p<0.01, Ad.mda-7 plus Ad.sgrp170 versus Ad.mda-7 or Ad.sgrp170). The results shown are from a representative two experiments.

FIG. 15 illustrates intratumoral delivery of Ad.mda-7 and Ad.sgrp170 promotes antigen-specific immune response. A. Establishment of TRAM-C2 tumor cell line stably expressing OVA (C2-OVA). TRAMP-C2 cells were transduced with pcDNA-OVA using FuGENE transfection reagent and selected in G418 (1 mg/ml)-containing medium. Expression of OVA was analyzed using RT-PCR assays. Primers of GAPDH were used as an internal control. B. Increased antigen-specific CTL frequency in mice following the combined therapies. Splenocytes were isolated from mice one week or three weeks after the last treatment. Cells were stimulated with OVA₂₅₇₋₂₆₄ (top) or mitomycin C-treated C2 cells at a ratio of 20:1 (bottom). IFN-γ production was measured using an ELISPOT assay (*p<0.01, Ad.mda-7 or Ad.sgrp170 versus control; **p<0.02, Ad.mda-7 plus Ad.sgrp170 versus Ad.mda-7 or Ad.sgrp170). Data (mean±s.d.) are representative of two separate experiments in which 3 mice of each group were analyzed. C. Co-injection of Ad.sgrp170 down-regulates Ad.mda-7 treatment induced IL-4 production in antigen-specific T-cells. Splenocytes were isolated from mice following treatment and subjected to ELISPOT assay for measuring OVA-stimulated IL-4 production (*p<0.01, Ad.mda-7 versus Ad.sgrp170 or Ad.mda-7 plus Ad.sgrp170). D. Enhanced cytolytic activity of effector T-cells in mice treated with the combined therapies. Mice with established C2 tumors were treated with Ad.mda-7, Ad.sgrp170 or Ad.mda-7 plus Ad.sgrp170 or left untreated. Splenocytes from the treated mice were harvested one week or three weeks after the last injection. Cells were re-stimulated with OVA₂₅₇₋₂₆₄ (SIINFEKL) in vitro for 5 d in the presence of IL-2 (10 U/ml). The CD8⁺ T-cells at different E:T ratios in triplicate were analyzed for cytotoxic activity using ⁵¹Cr-labeled C2-OVA as targets. Data are representative of three experiments.

FIG. 16 illustrates CD8⁺ T-cells contribute to the antitumor activities mediated by the combined therapies in vivo. A. Depletion of CD8⁺ T-cell subset abolishes antitumor immunity. Male C57BL/6 mice (n=6) with established C2-OVA tumors were depleted of CD4⁺, CD8⁺ T-cells by i.p. injection of GK1.5, 2.43 mAb respectively. Isotype-matched antibodies were used as controls. (CD8 depletion versus IgG control, p<0.01). B. The combined in situ therapies results in a tumor-specific immune response. Mice established with C2-OVA tumors were treated with Ad.mda-7 in combination with Ad.sgrp170 as described. C2-OVA tumor free mice following the combined therapies were rechallenged with parental TRAMP-C2 tumor (3×10⁶ cells) in contralateral side.

FIG. 17 illustrates antitumor immunity remains intact following separate administration of Ad.mda-7 and Ad.sgrp170. A. Treatment scheme for the modified combinational therapies. B. Injection of Ad.mda-7 and Ad.sgrp170 either together or separately generates a comparable antitumor response. Mice with established C2 tumors were treated with Ad.mda-7, Ad.sgrp170 together with Ad.mda-7 (T). One group of mice was treated with Ad.mda-7 and Ad.sgrp170 separately on different days (S). C. Both therapeutic regimens elicit similar levels of antigen-specific T-cells. Splenocytes isolated from mice one week after the last treatment and stimulated with OVA₂₅₇₋₂₆₄. IFN-γ production was measured using an ELISPOT assay (* and **p<0.01, versus control). D. T-effector cells from mice treated with Ad.mda-7 and Ad.sgrp170 together displayed an increased cytolytic activity compared with cells from mice treated with the two therapeutic agents separately. Splenocytes from the treated mice were re-stimulated with OVA₂₅₇₋₂₆₄ in vitro and assayed for the cytolytic activity using chromium release assays. OVA₂₅₇₋₂₆₄-pulsed C2 cells were used as targets. *p<0.01, Ad.mda-7+Ad.sgrp170(T) versus Ad.mda-7+Ad.sgrp170(S).

FIG. 18: Proof-of-principle showing the systemic delivery of mda-7/IL-24 (Ad.mda-7) into the prostate of Hi-Myc mouse. The preparation of MBs/Ad complex and US procedure is outlined in the protocol section. At the end of the experiments the prostate was dissected and preserved in formalin after which embedded paraffin sections were made and immunohistochemistry was performed to detect the transgene delivery of mda-7/IL-24.

FIG. 19. Microbubble-complexed GST-MDA-7 and ultrasound (US) eradicates human pancreatic cancer xenografts in nude mice. Human pancreatic cancer cell AsPC-1 was intraperitoneally injected into athymic nude mice resulting in development of tumors in the pancreatic bed. The animals were treated with microbubble-complexed GST-MDA-7 with or without US. In the absence of US the tumors continued to grow, while US treatment resulted in the disappearance of the tumor. Photographs have been taken after dissection of animals A: GST/microbubble without US B. GST with US. C: GST MDA-7 treatment i.p directly D. GST MDA-7/microbubble without US E. GST MDA-7/microbubble with US F. Western blot showing release of GST-MDA-7 in the tumor tissue only in the presence of US.

### DETAILED DESCRIPTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a cell” is a reference to one or more cells and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

### I. Introduction

The inventors have surprisingly discovered that viruses can be delivered systemically to an immunocompetent animal in microbubble suspensions. The suspensions are treated such that the suspensions lack virus on the surface of the suspensions. Once the suspensions have become systemic in the animal, they can be disrupted in a tissue-specific manner, thereby releasing virus only in a specific location. Notably, suspension of microbubbles in which virus was not removed from the surface of the suspension did not result in significant systemic delivery. Without intending to limit the scope of the invention, it is believed that inclusion of virus on the surface of the microbubble suspension (for example, resulting when the suspension is formed) raises an immune system response in immunocompetent animals that prevents successful delivery of the virus to a desired location in the animal. Therefore, the inventors have found that removal of virus from the surface of the suspension (e.g., by an initial incubation with complement, thereby removing the virus on the surface of the microbubbles) results in significantly improves systemic delivery. Once systemic delivery of the microbubbles is achieved, the microbubbles can be disrupted locally (e.g., with ultrasound), thereby releasing virus at only a desired location.

Provided immediately below is a “Definition” section, where certain terms related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

### DEFINITIONS

“PEG-3” refers to Progression Elevated Gene-3.

“mda-7” is Melanoma differentiation-associated gene-7/interleukin-24 (mda-7/IL-24). Mda-7 is a novel member of the IL-10-related cytokine gene family. It is a cancer-specific, apoptosis-inducing gene with broad-spectrum antitumor activity.

“Grp170” is 170 kDa glucose-regulated protein. Grp170 is an endoplasmic reticulum resident protein that shares some sequence homology with both the hsp70 and hsp110 heat shock protein (hsp) families, yet is representative of a third and unique family of stress proteins.

The term “immunocompetent” as used herein refers to the ability to produce a normal immune response, i.e., antibody production and/or cell-mediated immunity, following exposure to an antigen. In an immunocompetent system, there are mature B cells and T cells capable of recognizing and responding to an antigen. An immunocompetent animal has a functional thymus. For example, nude mice are not immunocompetent. Immunocompetent systems are capable of immunoglobulin and T-cell receptor gene recombination. For example, animals and human having mutated or knockout RAG1 and/or RAG2 genes are not immunocompetent because immunoglobulin and T-cell receptor gene recombination are compromised.

### I. Microbubble/Viral Suspensions

The methods of the invention include administering a virus (including but not limited to a selectively replicating virus) that is encompassed in a suspension of microbubbles. The microbubbles carry the virus to a site in the subject where the virus is released from the microbubble. In some embodiments, microbubbles are about 0.1 to 10.mu. in diameter and in a fluid medium. In one embodiment, the microbubble diameter is about 2.5 to 4 μm. This is small enough to prevent entrapment within the pulmonary capillary bed (ranging from 5 to 8 μm in diameter), but big enough to entrap and protect viral vectors, e.g., adenovirus, from the environment. In one embodiment, the microbubbles contain a blood-insoluble gas. Generally, any blood-insoluble gas which is nontoxic and gaseous at body temperature can be used. The insoluble gas should have a diffusion coefficient and blood solubility lower than nitrogen or oxygen, which diffuse in the internal atmosphere of the blood vessel. Examples of useful gases are the noble gases, e.g. helium or argon, as well as fluorocarbon gases and sulfur hexafluoride. In some embodiments, perfluorocarbon gases, such as perfluoromethane, perfluoroethane, perfluoropropane, perfluorobutane, and perfluoropentane, are used. It is believed that the cell membrane fluidizing feature of the perfluorobutane gas enhances cell entry for drugs on the surface of bubbles that come into contact with denuded vessel surfaces, as described further below.

In one embodiment, the microbubbles contain high-molecular weight gasses with less solubility and diffusivity, which improves microbubble persistence and allows passage through the microcirculation. In another embodiment, microbubbles are injected in peripheral veins, and the microbubbles can re-circulate through the systemic circulation numerous times, surviving for several minutes within the bloodstream. In addition, it is believed that the microbubbles protect the viruses from rapid degradation by the immune system, thus allowing for intravenous injection rather than direct target organ delivery by catheter-based approaches or operative bed injection. This is beneficial for cancer gene therapy of potentially inaccessible tumors, because the microbubbles may also limit the amount of inflammatory response to the viruses and may allow repeated injections. Targeting ligands on the surface of microbubbles permit the selective accumulation of these particles in the areas of interest, such as up-regulated levels of receptor/prognostic marker molecules on vascular endothelium or tumor cells.

The gaseous microbubbles can be stabilized by a fluid filmogenic coating, to prevent coalescence and to provide an interface for binding of molecules to the microbubbles. In some embodiments, the fluid is an aqueous solution or suspension of one or more components selected from proteins, surfactants, and polysaccharides. In some embodiments, the components are selected from proteins, surfactant compounds, and polysaccharides. Suitable proteins include, for example, albumin, gamma globulin, apotransferrin, hemoglobin, collagen, and urease. In one embodiment, the component is a human protein, e.g. human serum albumin (HSA). In another embodiment, as described below, a mixture of HSA and dextrose is used.

Conventional surfactants include compounds such as alkyl polyether alcohols, alkylphenol polyether alcohols, and alcohol ethoxylates, having higher alkyl (e.g. 6-20 carbon atom) groups, fatty acid alkanolamides or alkylene oxide adducts thereof, and fatty acid glycerol monoesters. Surfactants particularly intended for use in microbubble contrast agent compositions are disclosed, for example, in Nycomed Imaging patents U.S. Pat. No. 6,274,120 (fatty acids, polyhydroxyalkyl esters such as esters of pentaerythritol, ethylene glycol or glycerol, fatty alcohols and amines, and esters or amides thereof, lipophilic to aldehydes and ketones; lipophilic derivatives of sugars, etc.), U.S. Pat. No. 5,990,263 (methoxy-terminated PEG acylated with e.g. 6-hexadecanoyloxyhexadecanoyl), and U.S. Pat. No. 5,919,434.

Other filmogenic synthetic polymers may also be used; see, for example, U.S. Pat. No. 6,068,857 (Weitschies) and U.S. Pat. No. 6,143,276 (Unger), which describe microbubbles having a biodegradable polymer shell, where the polymer is selected from e.g. polylactic acid, an acrylate polymer, polyacrylamide, polycyanoacrylate, a polyester, polyether, polyamide, polysiloxane, polycarbonate, or polyphosphazene, and various combinations of copolymers thereof, such as a lactic acid-glycolic acid copolymer.

Such compositions have been used as contrast agents for diagnostic ultrasound, and have also been described for therapeutic applications, such as enhancement of drug penetration (Tachibana et al., U.S. Pat. No. 5,315,998), as thrombolytics (Porter, U.S. Pat. No. 5,648,098), and for drug delivery. The latter reports require some external method of releasing the drug at the site of delivery, typically by raising the temperature to induce a phase change (Unger, U.S. Pat. No. 6,143,276) or by exposing the microbubbles to ultrasound (Unger, U.S. Pat. No. 6,143,276; Klaveness et al., U.S. Pat. No. 6,261,537; Lindler et al., cited below, Unger et al., cited below; Porter et al., U.S. Pat. No. 6,117,858).

In one embodiment, the microbubbles are a suspension of perfluorocarbon-containing dextrose/albumin microbubbles known as PESDA (perfluorocarbon-exposed sonicated dextrose/albumin). Human serum albumin (HSA) is easily metabolized within the body and has been widely used as a contrast agent. The composition may be prepared as described in co-owned U.S. Pat. Nos. 5,849,727 and 6,117,858. Briefly, a dextrose/albumin solution is sonicated while being perfused with the perfluorocarbon gas. In one embodiment, the microbubbles are formed in an N₂-depleted or N₂-free, environment, typically by introducing an N₂-depleted (in comparison to room air) or N₂-free gas into the interface between the sonicating horn and the solution. Microbubbles formed in this way are found to be significantly smaller and stabler than those formed in the presence of room air. See e.g. Porter et al., U.S. Pat. No. 6,245,747.

The microbubbles encompass the virus, as described below. Generally, the microbubble suspension is incubated, with agitation if necessary, with the virus. The incubation can be carried out at room temperature, or at moderately higher temperatures, as long as the stability of the drug or the microbubbles is not compromised.

The viral suspension can be prepared using standard laboratory methods. Tissue culture cell lines or a suitable animal can be used to propagate the virus. Traditionally, cells have been used for viral propagation. The cells used have to be readily infected by the viruses. The cell lines then amplify the amount of virus, and in many cases die as a consequence of the viral infection producing characteristic cytopathic effects in the cell monolayer. After a virus is propagated in either cell culture or in a suitable animal, the infectivity titer of the virus material is obtained. The infectivity titer can be determined in vivo by inoculating increasing dilutions of the virus material to a susceptible host animal, such as laboratory mice. Based on mortality seen in different dilutions, the infectivity titer can calculated by suing either Reed-Muench or Karber formula. The infectivity titer is the reciprocal of highest dilution showing 50% mortality in the inoculated mice and expressed as LD₅₀/ml. In one embodiment, adenovirus is produced in HEK293 cells and infection titers are determined by plaque tittering on 293 cells. The virus can then be concentrated and purified, e.g., AdenoPACK Maxi columns. HEK293 cells are human embryonic kidney cells that contain and express the essential E1 region of the viral genome. This complementation, which is necessary because E1 is deleted in the vectors, does not occur in other cell types and is a safety feature for gene therapy purposes.

As noted herein, it is advantageous to remove virus on the surface of the microbubbles. Without intending to limit the scope of the invention, it is believed that microbubbles hide the virus from a competent immune system, which would otherwise recognize and kill the virus. Therefore, treatment of the microbubble suspensions to remove virus on the surface of the suspension may result in significantly improved systemic delivery of the microbubbles. Thus, in some embodiments, the microbubble suspensions are treated such that the suspensions lack virus on the surface of the suspensions. In one embodiment, the microbubble suspension is incubated with complement, thereby removing viruses on the surface of the microbubble suspension.

### II. Viruses

Any virus that has been used for gene therapy is suitable for use in this invention. Several different viruses have been used in gene therapy, including retroviruses, e.g., lentivirus, adenoviruses, adeno-associated viruses and herpes simplex viruses, can be used as described herein.

Adenoviruses are medium-sized (90-100 nm), nonenveloped (naked) icosahedral viruses composed of a nucleocapsid and a double-stranded linear DNA genome. Because of their size, they are able to be transported through the endosome (i.e. envelope fusion is not necessary). The virion also has a unique “spike” or fiber associated with each penton base of the capsid (see picture below) that aids in attachment to the host cell via the coxsackie-adenovirus receptor on the surface of the host cell. The adenovirus genome is linear, non-segmented double stranded (ds) DNA which is between 26 and 45 Kbp. This allows the virus to theoretically carry 22 to 40 genes. Although this is significantly larger than other viruses in its Baltimore group, it is still a very simple virus and is heavily reliant on the host cell for survival and replication. The viral genome has a terminal 55 kDa protein associated with each of the 5′ ends of the linear dsDNA, these are used as primers in viral replication and ensure that the ends of the virus' linear genome are adequately replicated. Adenoviruses are able to replicate in the nucleus of mammalian cells using the host's replication machinery.

Entry of adenoviruses into the host cell involves two sets of interactions between the virus and the host cell. Entry into the host cell is initiated by the knob domain of the fiber protein binding to the cell receptor. The two currently established receptors are: CD46 for the group B human adenovirus serotypes and the coxsackievirus adenovirus receptor (CAR) for all other serotypes. This is followed by a secondary interaction, where a specialized motif in the penton base protein interacts with an integrin molecule. It is the co-receptor av integrin interaction that stimulates internalization of the adenovirus. Binding to av integrin results in endocytosis of the virus particle via clathrin-coated pits. Once the virus has successfully gained entry into the host cell, the endosome acidifies and releases the virion into the cytoplasm. The virus is then transported to the nuclear pore complex whereby the adenovirus particle disassembles. Viral DNA is subsequently released which can enter the host cell's nucleus. Thus viral gene expression can occur and new virus particles can be generated.

In some embodiments, the virus is a modified adenovirus having a tissue or disease specific promoter (e.g., the PEG-3 promoter) operably linked to the E1A (and E1B) gene(s) which further comprises an additional active transcriptional unit expressing a heterologous (non-adenoviral) gene of interest. The gene of interest may encode a secreted product or a non-secreted product. Such modified viruses are referred to herein as “Terminator Viruses”. In some embodiments, the gene of interest is comprised in the E3 gene of adenovirus. Details of such viruses can be found in, e.g., US Patent Publication No. 2008/0213220.

A gene of interest may be, for example and not by way of limitation, a gene that augments immunity (in a subject to whom the virus is administered), such as IFN-.alpha., IFN-.beta., IFN-.gamma., IL-2, IL-4, IL-12 etc., a gene involved in innate immune system activation such as mda-5 (Kang et al., 2002 Proc Natl Acad Sci USA. 99(2):637-42), RIG-I (Heim, 2005, J. Hepatol. 42(3):431-3) etc., a gene that has an anti-cancer effect, including genes with anti-proliferative activity, anti-metastatic activity, anti-angiogenic activity, or pro-apoptotic activity, such as mda-7/IL-24 (Sarkar et al. (2002) Biotechniques Suppl: 30-39; Fisher et al. (2003) Cancer Biol Ther 2:S23-37), TNF-alpha (Anderson et al. Curr Opin Pharmacol (2004) 4(4):314-320), IFN-beta (Yoshida et al, (2004) Cancer Sci 95(11):858-865), p53 (Haupt et al. Cell Cycle (2004) 3(7):912-916), BAX (Chan et al. Clin Exp Pharmacol Physiol (2004) 31(3):119-128), PTEN (Sansal et al. J Clin Oncol (2004) 22(14):2954-63), soluble fibroblast growth factor receptor (sFGFR) (Gowardhan et al. (2004) Prostate 61(1):50-59), RNAi or antisense-ras (Liu et al. Cancer Gene Ther (2004) 11(11):748-756.), RNAi or antisense VEGF (Qui et al. Hepatobiliary Pancreat Dis Int (2004) 3(4):552-557), antisense or RNAi mda-9/syntenin (Sarkar et al. Pharmacol Ther (2004) 104(2):101-115) etc., a gene that renders an infected cell detectable, such as green fluorescent protein (or another naturally occurring fluorescent protein or engineered variant thereof), .beta.-glucuronidase, .beta.-galactosidase, luciferase, and dihydrofolate reductase, or a gene which enhances radiotherapy including but not limited to p53 (Haupt et al. Cell Cycle (2004) 3(7):912-916), GADD34 (Leibermann et al. Leukemia (2002) 16(4):527-41), the sodium iodide symporter (for thyroid cancer) (Mitrofanova et al. Clin Cancer Res (2004) 10(20):6969-6976), etc.

In further non-limiting embodiments a modified adenovirus having a PEG-3 promoter operably linked to the E1A and E1B genes and comprising an additional active transcriptional unit expressing a heterologous gene of interest may be utilized to deliver a therapeutic amount of an anti-inflammatory, anti-allergic or antiviral gene product either systemically or at a specific target site in a human subject or non-human animal. Non-limiting examples of such genes include IFN-α or IFN-β (Markowitz, Expert Opin Emerg Drugs (2004) 9(2):363-374) to treat an inflammatory condition or for anti-viral therapy (Suzuki et al. J Gastroenterol (2004) 39(10):969-974; Malaguamera et al BioDrugs. (2004) 18(6):407-413), Interferon Regulatory Factor-1 (IRF-1) for inflammation (Siegmund et al. Eur J Immunol (2004) 34(9): 2356-2364), mda-5 (Andrejeva et al., 2004 Proc Natl Acad Sci USA. 101(49):17264-9; Yoneyama et al; 2005, J. Immunol. 175(5):2851-8) and RIG-I (Meylan et al., 2005, Nature. 437(7062): 1167-72) for antiviral activity or stimulation of the innate immune system etc. In various non-limiting embodiments, a modified adenoviral vector may comprise, as a gene of interest, a gene having a product that enhances, in a subject having a cancer, the immune response of the subject to the cancer. Suitable genes of interest include, but are not limited to, genes encoding tumor-associated antigens recognized by the immune system, such as gp100, PSA, EGFR, CEA, HER-2/neu, C017-1a, MUC-1, gp72/CD55, gastrin, beta-HCG, alpha-fetoprotein, heat shock protein (gp96), etc. (Mocellin et al. (2004) Gastroenterology 127:1821-1837). Since inadequate or inhibitory T-cell costimulatory pathway signaling has been shown to restrict productive immune responses against cancer cells, genes of interest encoding costimulatory ligands such as B7-H3 (Luo et al. (2004) J Immunol 173(9):5445-5450), GM-CSF/IL-2 fusion protein (Stagg et al. (2004) Cancer Res 64(24): 8795-8799) etc. may be comprised in the modified adenoviruses of the invention.

In some embodiments, the gene of interest, located in (inserted into) the E3 or other suitable region of the adenoviral genome, is operably linked to a promoter element which is constitutively or inducibly active in the intended target cell (e.g., a cancer cell in a tumor to be treated). Suitable promoters include, but are not limited to, the cytomegalovirus immediate early promoter, the Rous sarcoma virus long terminal repeat promoter, the human elongation factor-1.alpha. promoter, the human ubiquitin c promoter, etc. (Colosimo et al. Biotechniques (2000) 29(2):314-318, 320-322, 324) and the PEG-3 promoter (U.S. Pat. Nos. 6,472,520 and 6,737,523; Su et al. (2000) Oncogene 19:3411-3421; Su et al. (2001) Nucleic Acids Res 29:1661-1671; provided the gene configuration having a direct repeat of two identical PEG-3 promoter DNA sequences separated by an intervening DNA does not undergo intramolecular recombination). It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. (Romano, Drug News Perspect (2004) 17(2):85-90). In specific non-limiting embodiments of the invention, the promoter may be selectively active in cancer cells, such as the prostate specific antigen gene promoter (O'Keefe et al. (2000) Prostate 45:149-157), the kallikrein 2 gene promoter (Xie et al. (2001) Human Gene Ther 12:549-561), the human alpha-fetoprotein gene promoter (Ido et al. (1995) Cancer Res 55:3105-3109), the c-erbB-2 gene promoter (Takakuwa et al. (1997) Jpn. J. Cancer Res. 88:166-175), the human carcinoembryonic antigen gene promoter (Lan et al. (1996) Gastroenterol. 111: 1241-1251), the gastrin-releasing peptide gene promoter (Inase et al. (2000) Int. J. Cancer 85:716-719). the human telomerase reverse transcriptase gene promoter (Pan and Koenman, 1999, Med Hypotheses 53:130-135), the hexokinase II gene promoter (Katabi et al. (1999) Human Gene Ther 10:155-164), the L-plastin gene promoter (Peng et al. (2001) Cancer Res 61:4405-4413), the neuron-specific enolase gene promoter (Tanaka et al. (2001) Anticancer Res 21:291-294), the midkine gene promoter (Adachi et al. (2000) Cancer Res 60:4305-4310), the human mucin gene MUC1 promoter (Stackhouse et al. (1999) Cancer Gene Ther 6:209-219), and the human mucin gene MUC4 promoter (Genbank Accession No. AF241535), which is particularly active in pancreatic cancer cells (Perrais et al. (2000) J Biol Chem 276(33):30923-30933).

A PEG-3 promoter of the invention may also be a nucleic acid molecule that is at least about 85 percent, at least about 90 percent, or at least about 95 percent identical to SEQ ID NO:1. The promoter sequences may be full length or active fragments thereof that retain the PEG-3 expression pattern. In some cases, the fragments can be at least 50, 100, 150, 200, 250, 300, 350, 400, 450, 500, 550, 600, 650, 700, 750, 800, and 850, base pairs of SEQ ID NO:1.

In some embodiments, a virus of the invention expresses a chaperone protein, with or without mda-7 or other heterologous protein. Chaperone proteins assist with the activity or function of other proteins in the cell. Many chaperone proteins are heat shock proteins or stress proteins, which prevent newly synthesized polypeptide chains and assembled subunits from aggregating. Chaperones can also assist in the assembly of nucleosomes from folded histones and DNA. Different stress proteins are very different in cellular functions and in their abilities to chaperone or bind antigens. In some embodiments, the virus expresses Grp 170. Grp 170 is a newly characterized stress protein and the largest endoplasmic reticulum (ER)-resident molecular chaperone, and is a highly diverged relative of the hsp70 family. See, e.g., Park J, Easton D P, Chen X, MacDonald I J, Wang X Y, Subjeck J R. Biochemistry 2003; 42:14893-902.

Grp170 has been shown to interact with transporter associated with antigen processing (TAP) translocated peptides and may be involved in polypeptide trafficking in the antigen presentation pathway. In particular, grp170 is capable of interacting with specific receptors on professional antigen-presenting cells (APCs) and shuttling antigens into the endogenous presentation pathway efficiently, which results in antigen presentation and tumor-specific immunity. In addition to promoting antigen cross-presentation, grp170 also acts as a ‘danger’ signal that stimulates phenotypic and functional maturation of dendritic cells. Without being bound by theory it is believed that the chaperone protein aids in presenting the antigen from the cancer cell that has been killed by the virus to the immune system, thereby creating a “vaccine effect” against cancer cells of that particular type. The immunochaperone grp170 may simultaneously deliver both an adjuvant effect for activation of innate immunity and antigenicity the virus, which would dramatically increase the intrinsic immunogenicity of tumor killing. Considering that tumor cells harbor a repertoire of unique, mutated-antigens as well as shared self-antigens, using the process of tumor cell death in combination with a potent immune activating agent/vaccine produced from within the same tumor cells may result in the permanent eradication of the primary tumor and metastases; thus preventing recurrence.

In one embodiment, the chaperone protein is encoded by the virus to target the tumor and augments the immune system such that a robust tumor-specific immune response is provided, leading to a significantly improved control of distant and secondary tumors. For example, intratumoral co-administration of secretable grp170 and the melanoma differentiation-associated gene-7/interleukin-24, which is a cancer-specific and apoptosis-inducing gene, each encoded by a virus, effectively and markedly suppress treated local prostate tumors (see Example 2).

In non-limiting embodiments, the tropism of the adenovirus may be altered to improve infection of adenovirus in target cells, e.g., cancer cells. The absence of the primary adenoviral receptor, i.e. the Coxsackie-Adenovirus Receptor (CAR), in target cells is a substantial obstacle to effective gene therapy, as it limits the access of cells to therapeutic virus. To overcome this obstacle, adenoviral vectors may be targeted to alternative cellular receptors by genetically modifying surface properties of the viral capsid. Specific modifications in the adenoviral capsid fiber can improve infectivity of prostate tumor cells by Triage viruses.

In one embodiment, a modified adenovirus may further comprise a virion fiber or hexon capsid protein modification to facilitate infection of target cells and/or enhance targeting of an adenovirus vector to specific cell types. Such viruses are referred to herein as “Triage Viruses”. Such capsid-modified adenoviruses are generically referred to in the literature as “infectivity enhanced” adenoviruses (Krasnykh et al. Cancer Res (2000) 60(24):6784-6787). Such modifications include but are not restricted to incorporation of targeting ligands within the capsid proteins.

In another embodiment, one or more heterologous targeting ligands may be incorporated within the fiber. Based on the three dimensional model of the fiber knob, targeting ligand may be inserted into the HI loop of the fiber (Ruigork et al. (1990) Mol Biol 215:589-596). This loop is flexible, exposed outside the knob, is not involved in fiber trimerization, and its variable length is different among Ad serotypes suggesting that insertions or substitutions do not substantially affect fiber stability (Krasnyk et al. (1996) J Virol 70:6839-6846; Douglas et al. (1996) Nature Biotech 14:1574-1578). In a specific non-limiting embodiment, two types of ligands may be introduced into the HI loop of the fiber: (i) the sequence coding for an RGD peptide, CDCRGRDCFC (SEQ ID NO:2), known to target tumors by binding with high affinity to several types of integrins thus facilitating binding via fiber-RGD/integrin interaction independent of the adenoviral CAR receptor (Krasnykh et al. Cancer Res (2000) 60(24):6784-6787); and (ii) the sequence encoding a poly-lysine (pK7)-peptide (GSGSGSGSGS) (SEQ ID NO:3) incorporated at the C terminal of the fiber protein) permitting attachment and entry through heparin sulfate-containing receptors which also facilitate CAR-independent infection (Krasnykh et al. Cancer Res (2000) 60(24):6784-6787). It has been demonstrated that infectivity of adenoviruses with modified fiber structure as described provides higher infectivity in prostate cancer cells.

In further embodiments, the conditionally replicating adenoviral vector may be tropism-modified by altering the nature and properties of the hexon protein (Krasnyk et al. (1996) J Virol 70:6839-6846). The hexon protein is in greater than twenty-fold abundance than the fiber protein. The hexon protein may be modified to contain a small peptide ligand with high specificity for a cellular target. When expressed as a heterologous component of a hexon protein a small peptide ligand is presented on the surface of an adenovirus with high relative abundance. Peptide ligands when presented in this manner overcome potential lack of high affinity through increased avidity. Modification of hexon protein may be accomplished by genetic incorporation of DNA sequences coding for ligands into the hyper-variable regions of the hexon gene utilizing a suitable shuttle vector. In additional non-limiting embodiments, the fiber knob may be altered by genetic incorporation of alternate knob domains (Henry et al (1994) J Virol 68(6):5239-5246; Krasnyk et al. (1996) J Virol 70: 6839-6846).

To overcome the limitations associated with infectivity due to reduced CAR, in some embodiments adenovirus having genetically modified infectivity tropisms may be used to provide enhanced infectivity and improved oncolytic potency. For example, in some embodiments, the viruses are altered such that they bind to non-CAR receptors.

In yet another embodiment, the tropism of the microbubble is changed by incorporating an agent with specific tropism into microbubbles. In one aspect, the microbubbles include a PSMA inhibitor, directed to the prostate specific membrane antigen (PSMA), to enhance the systemic targeting of the microbubbles to prostate cancer cells.

### III. Treatment of Disease

The methods of the invention may be used to target a variety of diseases and tissue types. In fact, the invention can be used to treat any condition for which the virus suspended in microbubbles provides treatment, protection or amelioration. In one embodiment, the methods are used to target breast cancer, bronchial cancer, lung cancer, prostate cancer, colon cancer, rectal cancer, hepatic carcinoma, urogenital cancer, ovarian cancer, testicular carcinoma, osteosarcoma, chondrosarcoma, gastric cancer, pancreatic cancer, nasopharyngeal cancer, thyroid cancer, neuroblastoma, astrocytoma, glioblastoma multiforme, melanoma, hemangiosarcoma, an epithelial cancer, a non-epithelial cancer such as squamous cell carcinoma, leukemia, lymphoma, and cervical cancer. In another embodiment, the methods may be used to target solid tumors or metastatic tumors.

The invention further provides a method for producing a cytopathic effect in a cell comprising administrering a virus in a microbubble suspension is administered, the virus is released at a desired location (e.g., by bursting the bubbles with ultrasound, and infecting the target cell(s) with a modified adenovirus according to the invention. Types of cytopathic effects include a decrease in cell proliferation, a decrease in cell metabolism, and/or cell death. The cell may be a cancer cell of for example, a nasopharyngeal tumor, a thyroid tumor, a central nervous system tumor (e.g., a neuroblastoma, astrocytoma, or glioblastoma multiforme), melanoma, a vascular tumor, a blood vessel tumor (e.g., a hemangioma, a hemangiosarcoma), an epithelial tumor, a non-epithelial tumor, a blood tumor, a leukemia, a lymphoma, a cervical cancer, a breast cancer, a lung cancer, a prostate cancer, a colon cancer, a hepatic carcinoma, a urogenital cancer, an ovarian cancer, a testicular carcinoma, an osteosarcoma, a chondrosarcoma, a gastric cancer, or a pancreatic cancer. The cell may be a cancer cell in a human or a non-human animal subject. To achieve infection, the amount of modified virus administered may be, but not by way of limitation, between about 1×10¹⁰ to 1×10¹³ pfu or at a multiplicity of infection (m.o.i.) of between about 10 and 5000 virus particles, between about 10 and 1000 virus particles, or between about 100 and 1000 virus particles, per estimated cell (where the tumor volume can be estimated, the number of cells in the tumor may be estimated (e.g., a spherical tumor having a diameter of 1 cm may be estimated to contain 10⁹ cells; see James et al. (1999) JNCI 91:523-528)), and the effective amount may be administered in a series of inoculations, for example, between 1 and 15 inoculations, or between about 3 and 12 inoculations, or between about 3 and 7 inoculations, each containing between about 1×10¹⁰ to 1×10¹² pfu or at a multiplicity of infection (m.o.i.) of between about 10 and 5000 virus particles, between about 10 and 1000 virus particles, or between about 100 and 1000 virus particles, per estimated cell. Where the modified adenovirus is administered to a subject, the mode of administration (inoculation) may be, but is not limited to, intra-tumor instillation, intravenous, intra-arterial, intrathecal, intramuscular, intradermal, subcutaneous, mucosal via pulmonary or other route, direct nasal installation, etc.

In one set of non-limiting embodiments, the invention provides for methods of using the modified adenoviral vectors of the invention to treat forms of cancer which are refractory to conventional therapies (“refractory cancers”), or to inhibit the proliferation of a cell of a refractory cancer, or to inhibit tumor growth and/or metastasis of a refractory cancer. In one non-limiting set of embodiments, a cancer which has not shown adequate clinical response to a treatment agent, or combination thereof, which is not a modified adenoviral vector of the invention, is considered such a refractory cancer. In another non-limiting set of embodiments, a cancer which overexpresses as antiapoptotic protein, such as but not limited to Bcl-2 or Bcl-xL, is such a refractory cancer. In a specific, non-limiting example, a refractory cancer is apoptosis-resistant and/or treatment resistant prostate cancer. In other specific, non-limiting examples, a breast cancer which overexpresses Bcl-2, a small-cell lung cancer which overexpresses Bcl-2, a non-small cell lung cancer which overexpresses Bcl-2, and a liver cancer which overexpresses Bcl-2, are each considered to be refractory cancers.

Accordingly the invention provides for a method of treating a subject suffering from a cancer, where the cancer is selected from the group consisting of breast cancer, lung cancer, prostate cancer, colon cancer, rectal cancer, hepatic carcinoma, urogenital cancer, ovarian cancer, testicular carcinoma, osteosarcoma, chondrosarcoma, gastric cancer, pancreatic cancer, nasopharyngeal cancer, thyroid cancer, neuroblastoma, astrocytoma, glioblastoma multiforme, melanoma, hemangiosarcoma, an epithelial cancer, a non-epithelial cancer such as squamous cell carcinoma, leukemia, lymphoma, and cervical cancer, comprising administering, the subject, an effective amount of a modified adenovirus according to the invention. In non-limiting embodiments of the invention, an effective amount of modified adenovirus may be between about 1×10¹⁰ to 1×10¹² pfu or a multiplicity of infection (m.o.i.) of between about 10 and 5000 virus particles, between about 10 and 1000 virus particles, or between about 100 and 1000 virus particles, per estimated cell, and the effective amount may be administered in a series of inoculations, for example, between 1 and 15 inoculations, or between about 3 and 12 inoculations, or between about 3 and 7 inoculations, each containing between about 1×10¹⁰ to 1×10¹² pfu or at a multiplicity of infection (m.o.i.) of between about 10 and 5000 virus particles, between about 10 and 1000 virus particles, or between about 100 and 1000 virus particles, per estimated cell.

Treating a subject suffering from a cancer means one or more of the following: decreasing tumor volume; decreasing rate of tumor growth; increasing survival; decreasing tumor grade; inhibiting metastasis (meaning inhibiting dissemination and/or growth/proliferation of metastatic cells), increasing time of survival, and/or improving quality of life (e.g., decreasing pain, increasing ability to perform activities).

The invention in further non-limiting embodiments provides for a method of treatment of various types of cancer cells involving combined treatment with a microbubble suspension of a Terminator or Triage Virus in combination with radio- or chemotherapeutic agents. PEG-3 promoter activity is enhanced by DNA damaging agents and ionizing radiation (Su et al. (1999) Proc Natl Acad Sci USA 96(26):15115-151120; Su et al. (2002) J Cell Physiol 192(1):34-44). Therefore enhanced viral replication leading to enhanced cytolysis of tumor cells may be achieved. Combination therapy includes but is not limited to simultaneous or serial treatment with a Terminator or Triage Virus embodied in instant invention and standard radiotherapy or chemotherapy regimes. Chemotherapy may include but is not limited to treatment with appropriate doses of chemotherapy agents such as Cisplatin, Adriamycin, Doxorubicin, Paclitaxel or other Taxol derivatives, etc. In an additional embodiment, specific targeting to an organ, tumor or tissue type or enhanced infectivity is obtained by utilizing an appropriate Triage Virus.

In further non-limiting embodiments, a combination of two or more Terminator or Triage Viruses suspended in microbubbles may be used for a method of treatment of a cancer or other disease state. In this embodiment two or more Terminator or Triage Viruses expressing distinct genes of interest may be used in combination (administered concurrently or sequentially) for treatment in a human or non-human animal subject. Non-limiting examples of such combinations include treatment of a subject with two Terminator viruses, one expressing a gene of interest encoding IFN-alpha, IFN-beta, IFN-gamma, IL-2, IL-4, IL-12, RIG-I, mda-5 etc. and the other expressing a gene of interest encoding a tumor specific antigen or an immune accessory molecule such as Carcino-Embryonal Antigen (CEA), the B7.1 gene, lymphocyte homing receptor or HLA antigen gene.

In further non-limiting embodiments, Terminator or Triage Viruses expressing appropriate genes of interest may also be utilized to restore or boost the responsiveness of a subject to a specific form of conventional radio-, chemo- or immunotherapy. Non-limiting examples of such viruses contain a gene of interest which encodes the EGFR (Epidermal Growth Factor Receptor) or related variants such as the Her-2/neu receptor thereby enhancing a subject's responsiveness to therapies such as Herceptin in breast cancer patients or other anti-EGRF therapies such as Gefitinib (Iressa, ZD1839) an EGFR specific tyrosine kinase inhibitor or the tyrosine kinase inhibitor NVP-AEE788 (AEE788) which blocks both the EGF and VEGF signaling pathways. Viruses containing a gene if interest encoding the androgen receptor (AR) may be used to enhance or restore responsiveness to anti-androgen therapy in androgen refractive forms of prostate cancer. In further embodiments, Triage Viruses that target expression to specific tissues such as breast or prostate and in addition, restore responsive therapeutic targets such as EGFR or AR may be utilized to localize and enhance the efficiency of a particular form of radio-, chemo- or immunotherapy.

The methods of the invention include systemic delivery of virus via microbubble suspensions. Once the suspension is administered the microbubbles can be disrupted locally to releasing virus at a desired location. In one embodiment, the microbubbles are disrupted using ultrasound. Without being bound by theory, it is believed that ultrasound-targeted microbubble destruction (UTMD) enables focal release of entrapped materials as well as the creation of small shock waves that increase cellular permeability.

Gas filled microbubbles have been conventionally used as contrast agents for diagnostic ultrasound. They have also been described for therapeutic applications, such as enhancement of drug penetration (Tachibana et al., U.S. Pat. No. 5,315,998), as thrombolytics (e.g. Porter, U.S. Pat. No. 5,648,098), and for drug delivery. Reports of use of microbubbles for drug delivery have generally described the use of some external method of releasing the drug from the microbubbles at the site of delivery, by, for example, raising the temperature to induce a phase change (Unger, U.S. Pat. No. 6,143,276) or exposing the microbubbles to ultrasound (Unger, U.S. Pat. No. 6,143,276; Klaveness et al., U.S. Pat. No. 6,261,537; Lindler et al., Echocardiography 18(4):329, May 2001, and Unger et al., Echocardiography 18(4):355, May 2001; Porter et al., U.S. Pat. No. 6,117,858).

### EXAMPLES

The following examples are offered to illustrate, but not to limit the claimed invention.

### Example 1

A major challenge for effective gene therapy is the ability to specifically deliver nucleic acids and potentially toxic gene products directly into diseased tissue. Progress in gene therapy has been hampered by concerns over the safety and practicality of viral vectors, particularly for intravenous delivery, and the inefficiency of currently available non-viral transfection techniques [12]. Viruses are appealing delivery vectors because of their ability to efficiently transfer genes with sustained and robust expression. Recombinant Ads are one of the most common gene transfer vectors utilized in human clinical trials, but systemic administration of this virus is thwarted by host innate and adaptive antiviral immune responses which can limit and/or preclude repetitive treatment regiments [13].

The quest for novel, safe and more efficient systemic gene delivery systems has recently highlighted ultrasound (US) contrast agents (microbubbles) as a potential candidate for enhancing delivery of molecules to target tissue [14-17]. Currently used US contrast agents (microbubbles) contain high-molecular weight gasses with less solubility and diffusivity, which improves microbubble persistence and allows passage through the microcirculation. Microbubbles (MB) can be injected in peripheral veins, because the more robust bubbles can re-circulate through the systemic circulation numerous times, surviving for several minutes within the bloodstream [17, 18]. The ideal MB diameter most likely is between 2.5 to 4 μm. This is small enough to prevent entrapment within the pulmonary capillary bed (ranging from 5 to 8 μm in diameter), but big enough to entrap and protect viral vectors such as Ad from the environment.

The feasibility of site-specific gene delivery mediated by diagnostic US using Ad-GFP encapsulated in commercially available US contrast agents was previously demonstrated in vitro and in vivo [12]. An additional goal of our previous study was to determine if incubation of the microbubbles with complement could improve specificity of viral transgene transduction to the target tissue/organ allowing a simplified approach to encapsulation of the viral vectors with commercially available contrast agents. In the current investigation we tested a US contrast agent provided by Targeson, Inc (San Diego, Calif.) and the portable SonoSite Micro-Maxx ultrasound platform (SonoSite, Inc., Bothell, W A) equipped with a L25 linear array transducer. Targeson's agents are lipid-encapsulated perfluorocarbon microbubbles with a mean diameter of 2.5 μm that can be used in a wide variety of animal models, and are compatible with virtually all ultrasound scanners. The gas-filled microspheres effectively lower the energy threshold for non-thermal cavitation. This allows diagnostic transducers operating within the energy levels mandated by the FDA to be used for drug/gene delivery. Ultrasound-targeted microbubble destruction (UTMD) enables focal release of entrapped materials as well as the creation of small shock waves that increase cellular permeability [19]. In addition, the microbubbles protect the viruses from rapid degradation by the immune system, thus allowing for intravenous injection rather than direct target organ delivery by catheter-based approaches or operative bed injection [12, 17]. This is particularly important in cancer gene therapy of potentially inaccessible tumors because the microbubbles may also limit the amount of inflammatory response to the viruses and may allow repeated injections.

In this example, we provide proof-of-principle for two essential components of this process, a site-specific gene delivery approach mediated by diagnostic US generated by a portable platform that works efficiently in vivo in combination with Ads delivering a highly effective, broad-based cancer gene therapeutic mda-7/IL-24. Evidence is provided that this combination has profound effects in animal models containing therapy-resistant human prostate cancer cells.

Targeson microbubbles and a SonoSite portable MicroMaxx ultrasound (US) platform efficiently targets Ad-GFP viruses to tumors.

The feasibility of in vivo gene delivery mediated by diagnostic US using Ad-GFP encapsulated in a series of commercially available US contrast agents was also previously demonstrated [12]. In the current investigation, we tested a different US contrast agent available from Targeson, Inc (San Diego, Calif.) and the portable SonoSite Micro-Maxx ultrasound platform (SonoSite, Inc., Bothell, Wash.) equipped with a L25 linear array transducer. Targeson's agents are lipid-encapsulated perfluorocarbon microbubbles with a mean diameter of 2.5 μm that can be used in a wide variety of animal models, and are compatible with virtually all ultrasound scanners [20]. Targeson agents are normally sold as already reconstituted contrast agents that are stable for three months from arrival, and for this study we obtained a custom made freeze-dried Targeson contrast agent (perfluorocarbon microbubbles, encapsulated by a lipid monolayer and poly(ethyleneglycol) stabilizer) to be reconstituted with the viruses as previously described [12].

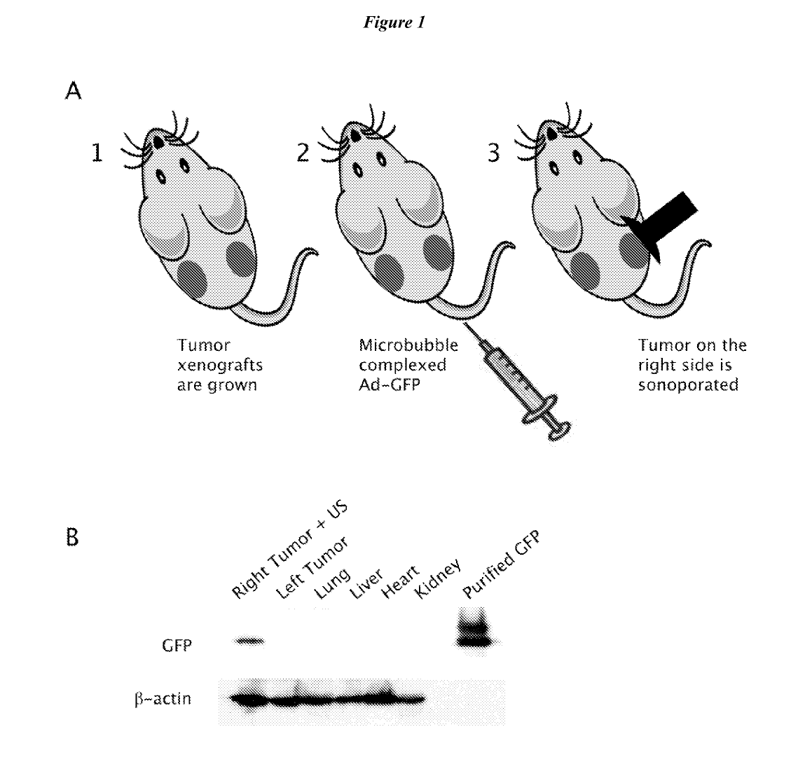

To confirm the ability of the lyophilized Targeson US contrast agent to deliver viruses efficiently and specifically to defined sites in vivo, we performed a pilot study in which tumor xenografts were established in both flanks of athymic nude mice by injecting each site with 2×10⁶ DU-145 human prostate carcinoma cells (FIG. 1A). The DU-145 tumor-bearing nude mice (n=10) were then injected in their tail vein with 100 μL of US contrast agent that was reconstituted with Ad-GFP or water as control. A portable SonoSite Micro-Maxx ultrasound platform (SonoSite, Inc., Bothell, Wash.) equipped with a L25 linear array transducer set at 0.7 Mechanical Index (MI), 1.8 MPa for 10 min was used to sonoporate only the tumor implanted on the right side (FIG. 1A). Mice were sacrificed 96 hr after treatment and tumors (right and left side), lung, heart, liver and kidney were harvested and snap frozen. FIG. 1B shows the specific delivery to the right tumor as evidenced by expression of the green fluorescence protein (GFP) in an immunoblot in which total protein extracts were run on a 10% SDS-PAGE. As a GFP control, we ran a GST-GFP fusion protein. Protein gel loading was normalized using β-actin as a control.

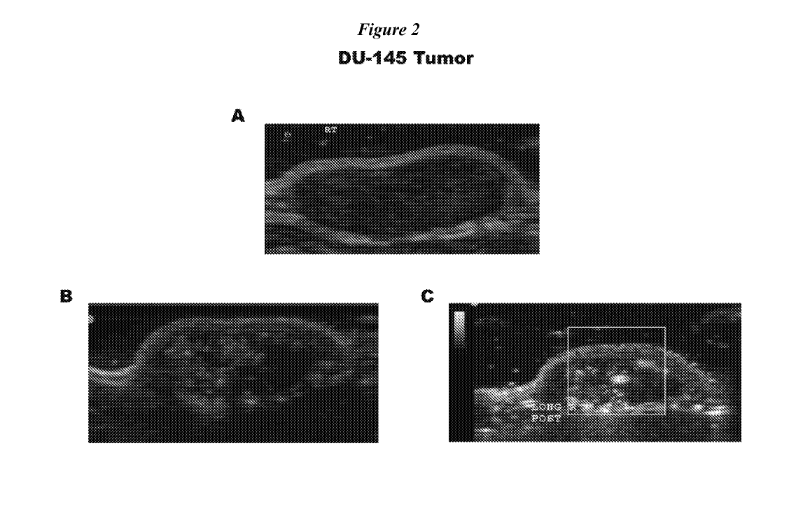

US-targeted microbubble destruction (UTMD) enables focal release of entrapped materials as well as the creation of small shock waves that are visualized as an enhancement of the image on the US scanner. FIG. 2, panel A depicts the B-mode US imaging of a sonoporated tumor before injection with the microbubble/Ad-GFP complex contrast agent. FIG. 2, panel B shows the B-mode ultrasound imaging of the same sonoporated tumor following microbubble/Ad-GFP complex injection. The image enhancement of the targeted tumor from cavitation of the microbubbles within the US field of view is clearly discernable indicating that the US settings are efficient in targeting microbubble destruction.

Microbubble Assisted Ad.Mda-7 Gene Delivery Inhibits Du-145 Human Prostate Cancer Growth In Vivo.

In vitro and in vivo Ad-mediated gene transfer of the human mda-7/IL-24 gene (Ad.mda-7) potently suppresses the growth of human cancer cells with no apparent toxicity to normal cells [6, 8, 9, 21-30]. Repeated intratumoral administration of Ad.mda-7 to tumor xenografts of various histological origin results in growth suppression via induction of apoptosis and anti-angiogenic mechanisms [4-9, 25, 28, 31]. Additionally, mda-7/IL24 induces a profound “bystander” antitumor effect resulting in tumor growth suppression not only in the treated tumors, but also in untreated distant tumors [6, 8-11, 22, 23, 25, 27, 32-36]. Although these results have been encouraging, this approach is limited since systemic delivery of Ad for treatment of disseminated cancer have not shown significant efficacy.

A novel systemic delivery approach to target Ad release in a site-specific manner that consists of Ad incorporated in microbubbles combined with diagnostic US was employed [12]. Proof-of-principle for this strategy comes from studies using Ad to systemically deliver the GFP gene in a tissue specific manner [12]. Because mda-7/IL-24 has shown significant potential as a selective and effective anticancer agent in multiple animal model studies and in a Phase I intratumoral gene therapy trial in patients with advanced solid cancers [6-9, 21-23, 28], we tested the capacity of this approach to deliver Ad expressing this novel cytokine in prostate adenocarcinoma nude mouse xenograft models. For these studies, we used DU-145 human prostate carcinoma cells and DU-145 cells genetically engineered to express elevated levels of Bcl-x_{L} (DU-Bcl-xL) [4], which is a common event in advanced prostate cancer and provokes resistance to multiple chemotherapeutic agents and to mda-7/IL-24 [2-5]. The therapeutic arm of this work included two different viral constructs to deliver mda-7/IL-24, Ad.mda-7, a nonreplicating Ad similar to the one used in Phase I clinical trials [26], and the CTV, a conditionally replication competent Ad capable of expressing mda-7/IL-24 that has been previously shown to completely eradicate not only primary breast, prostate and melanoma tumors but also distant tumors by intratumoral injections in a nude mouse model [11, 33, 34, 37].

To test this new therapeutic approach for tumor delivery, DU-145 or DU-Bcl-xL tumor xenografts were established on both flanks of nude mice by injecting 2×10⁶ cells in each side of the animals. DU-145 and DU-Bcl-xL tumor bearing nude mice (n=10 each group) were then injected in their tail vein with 100 μL of US contrast agent that was reconstituted with Ad-GFP or water as control. Additional DU-145 and DU-Bcl-xL tumor control nude mice (n=10 each group) were injected in the tail vein with 100 μL of Ad.mda-7 or the CTV (Ad.PEG-E1A-mda-7) without US contrast agent. Alternatively, tumor-bearing animals were injected in their tail vein with 100 μL of US contrast agent that was reconstituted with Ad.mda-7 or the CTV. A portable SonoSite Micro-Maxx US platform (SonoSite, Inc., Bothell, Wash.) equipped with a L25 linear array transducer set at 0.7 Mechanical Index (MI), 1.8 MPa for 10 min was used to sonoporate the tumor implanted on the right-side. In this study, gene therapy treatments were started ten weeks after the injection of the cell lines, when tumors reached an approximate volume of 150-200 mm³. Mice were injected once a week for four weeks for a total of four treatments. Mice were sacrificed two weeks after the end of the treatments to determine whether tumor suppression was reversible or irreversible. At the end of the study tumors (right and left flank), lung, heart, liver and kidney were harvested and snap frozen using liquid nitrogen.

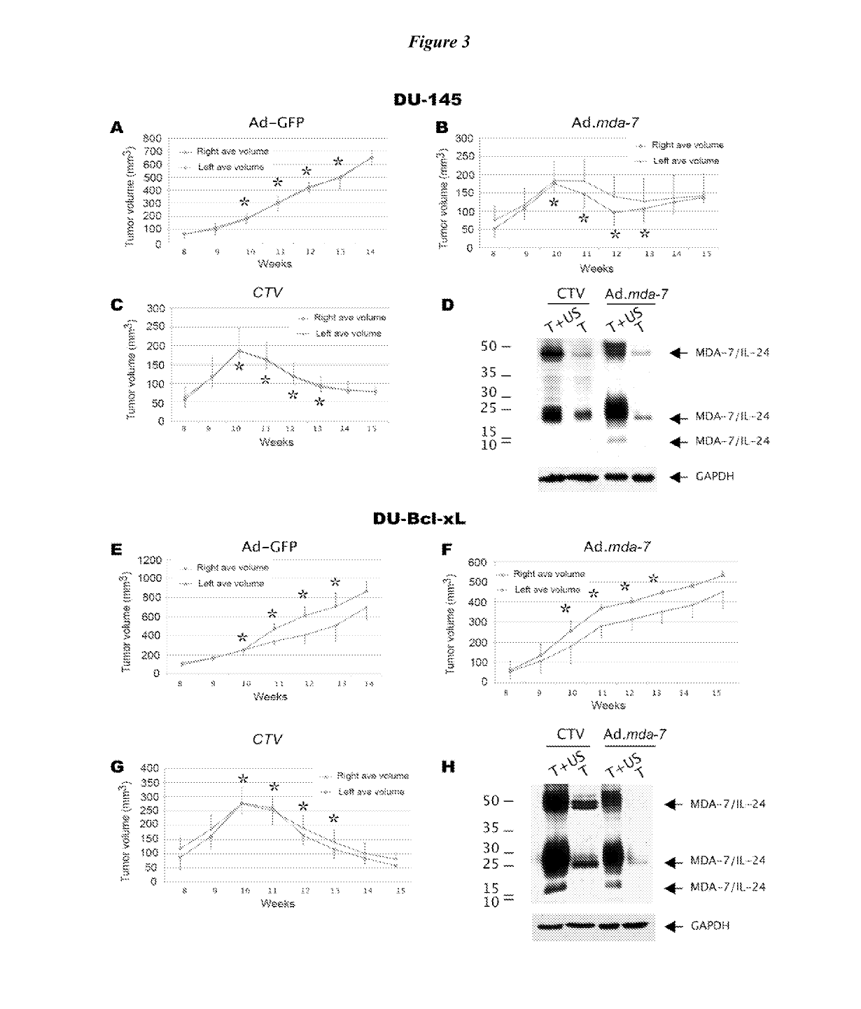

US of Ad-GFP microbubble complexes in the right side tumor resulted in progressive growth of the tumors on both flanks (FIGS. 3A and E). The results shown in FIG. 3 represent the average tumor volumes measured in a minimum of 7 mice for each mda-7/IL-24 group and a minimum of 5 mice for each control GFP group. All the mice were injected in the tail vein with the microbubble/Ad complexes and only the tumor on the right flank was sonoporated. Interestingly, we observed that microbubble-mediated Ad.mda-7 gene therapy inhibited the growth of DU-145 prostate tumor xenografts during the treatment regimen (FIG. 3B), while the CTV microbubble-mediated gene therapy resulted in a steady progressive tumor regression that lasted an additional two weeks post-treatment (FIG. 3C).

As predicted from previous studies [4, 5, 11], Ad.mda-7 was ineffective in causing a therapeutic response in tumor xenografts on either flank developed from DU-Bcl-xL cells (FIG. 3F). In contrast, the conditionally replication competent CTV (Ad.PEG-Prom-mda-7) elicited a sustained growth inhibition of the therapy resistant DU-Bcl-xL tumor xenografts (FIG. 3G). A Western blot analysis of total protein extracts from the harvested tumors showed expression of MDA-7/IL-24 protein in both the tumor samples implanted on the right and left flank (FIGS. 3D and H) validating the “bystander” effects of MDA-7/IL-24 previously reported [11, 33, 35-37]. In the case of the CTV, this amplified expression of MDA-7/IL-24 in the non-injected left tumor may also reflect secondary viral infection by the CRAD [11]. GAPDH expression was used to confirm equal loading of the gel. No tumor regression was observed in mice bearing DU-145 and DU-Bcl-xL control tumors when injected intravenously with comparable doses of unprotected Ad.mda-7 and CTV viruses (Supplemental figures s1 and s2).

Microbubble Assisted CTV Gene Delivery Eradicates Prostate Cancer Growth in Vivo.

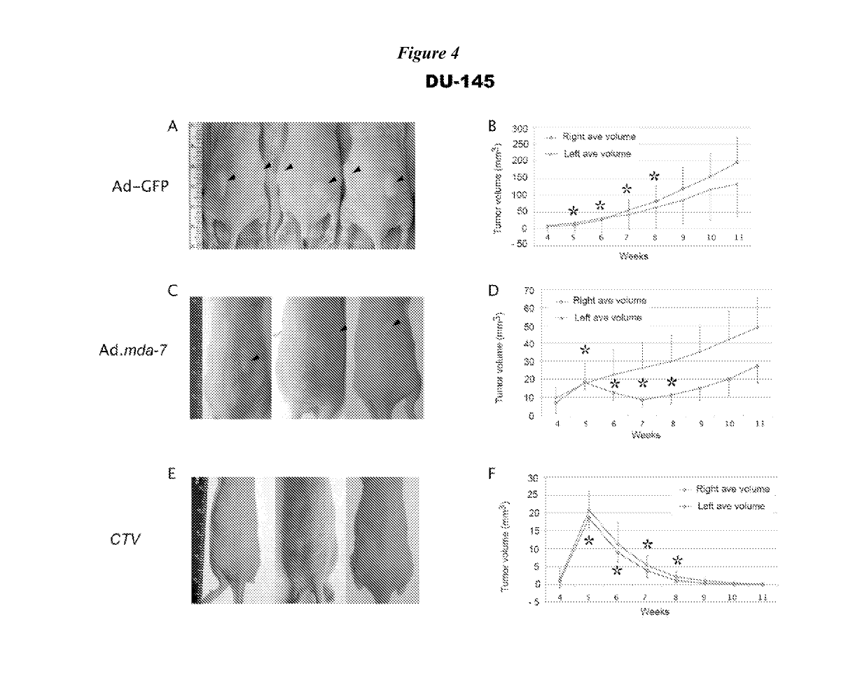

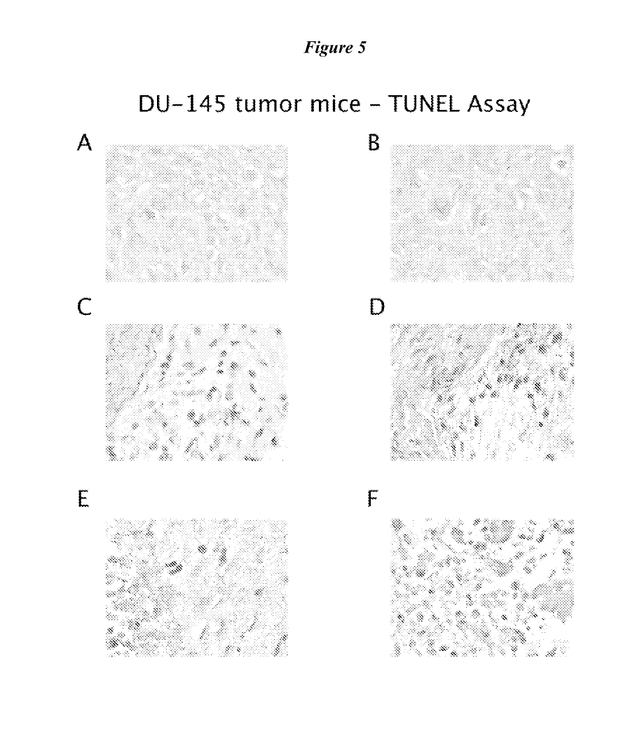

Based on the initial positive results obtained with the CTV-microbubble delivery approach, further experiments were performed in nude mice. Tumor xenografts were again established on both flanks of nude mice by injecting 1.5×10⁶ DU-145 or DU-Bcl-xL cells. After palpable tumors developed in 5 weeks (25-50 mm̂3), four injections of the various microbubble/Ad complexes into the tail vein once a week (for a total of four weeks) with US for 10 min on the tumor on the right side were performed. No treatment was performed on the tumor xenografted on the left flank. Injection of Ad-GFP-microbubble and US in the right tumor plus US did not significantly impact tumor growth on either flank (FIGS. 4A and 4B). The data is presented as the average tumor volumes measured in at least 9 mice for each active experimental group, with 6 mice in the control Ad-GFP group. In the case of CTV-microbubble-treated animals the experiment was terminated after 6 weeks because DU-145 tumors on both sides showed regression after two injections, and with four injections tumors were completely eradicated (FIGS. 4E and 4F). Additionally, control GFP-transduced DU-145 tumor xenografts reached tumor volumes of 250-300 mm³ after similar treatment. Although Ad.mda-7-microbubble treatment visibly inhibited the growth of tumors on the sonoporated flank, it had some inhibitory effect on tumors on the left side, which was not statistically significant (FIGS. 4C and 4D). Interestingly, treatments with the CTV-microbubble complexes resulted in disruption of the tumor cyto-architecture, as visualized by Hematoxylin and Eosin staining (data not shown), which correlated with an increase in the number of TUNEL positive tumor cells in both sonoporated (FIG. 5E) and non-sonoporated tumors (FIG. 5F). Control tumors treated with Ad-GFP-microbubble complexes were mostly TUNEL negative, showing a few cells positive to the TUNEL colorimetric reaction as depicted in FIG. 5A (sonoporated tumor on the right flank) and FIG. 5B (untreated tumor). Additionally, DU-145 tumor xenografts treated with Ad.mda-7-microbubble complexes showed a higher percentage of TUNEL positive tumor cells (FIG. 5C) than the untreated left flank (FIG. 5D).

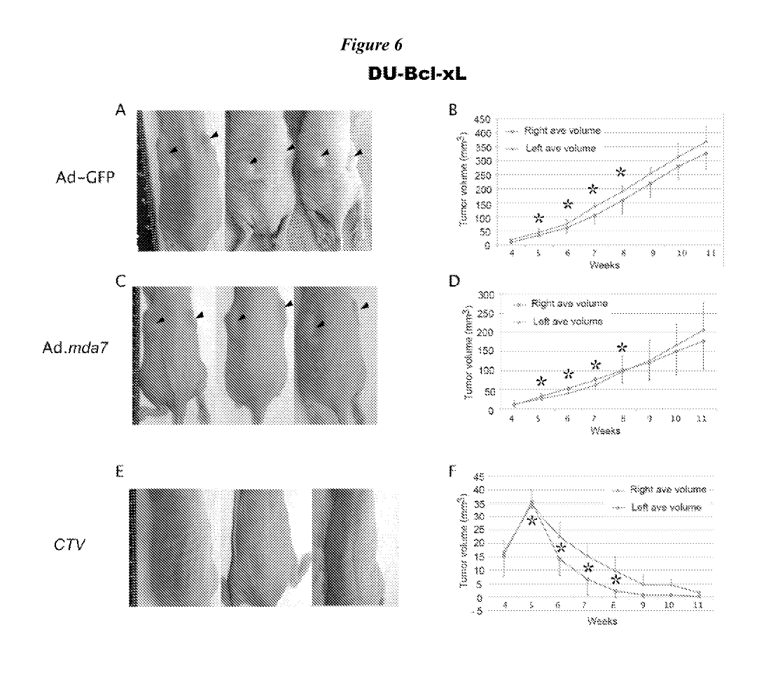

Experiments were next performed in the therapy resistant DU-Bcl-xL xenografted mice (FIG. 6). In this study mice with established xenografts on both flanks were injected in the tail vein with the microbubble/Ads complexes, and only the tumors on the right flank were sonoporated. Animals receiving the Ad-GFP-microbubble complexes plus US treatment showed no statistically significant effect on the growth of DU-Bcl-xL tumors (FIGS. 6A and B). The data presented is the average tumor volumes of 9 mice receiving therapeutic gene treatment and 6 mice receiving Ad-GFP treatment. The experiment was terminated after 6 weeks with injections of CTV-microbubble complexes because DU-Bcl-xL tumors on both sides showed regression after only two injections, and within four injections profound tumor regression was evident (FIGS. 6E and 6F). Additionally, three weeks after the last injections tumors were completely eradicated. Control Ad-GFP-microbubble complex treated DU-Bc1-xL tumor xenografts reached tumor volumes of 350-400 mm³ requiring the mice to be sacrificed as per our animal protocol.

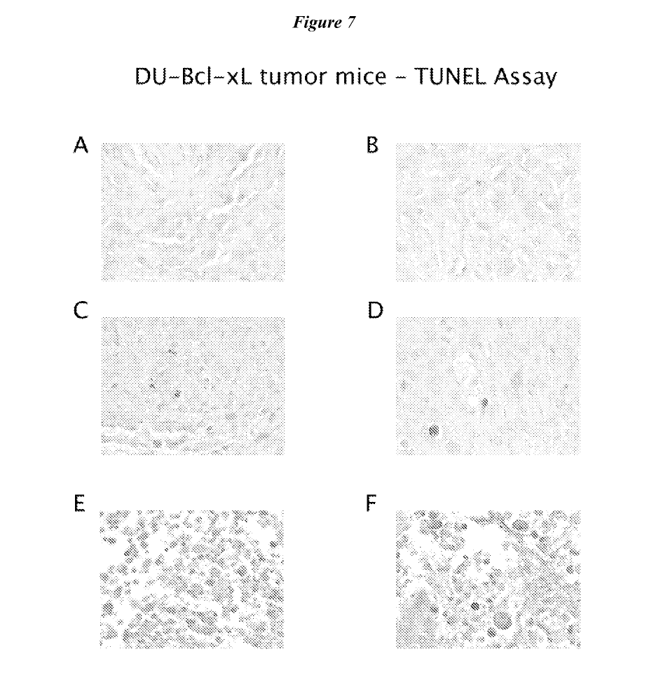

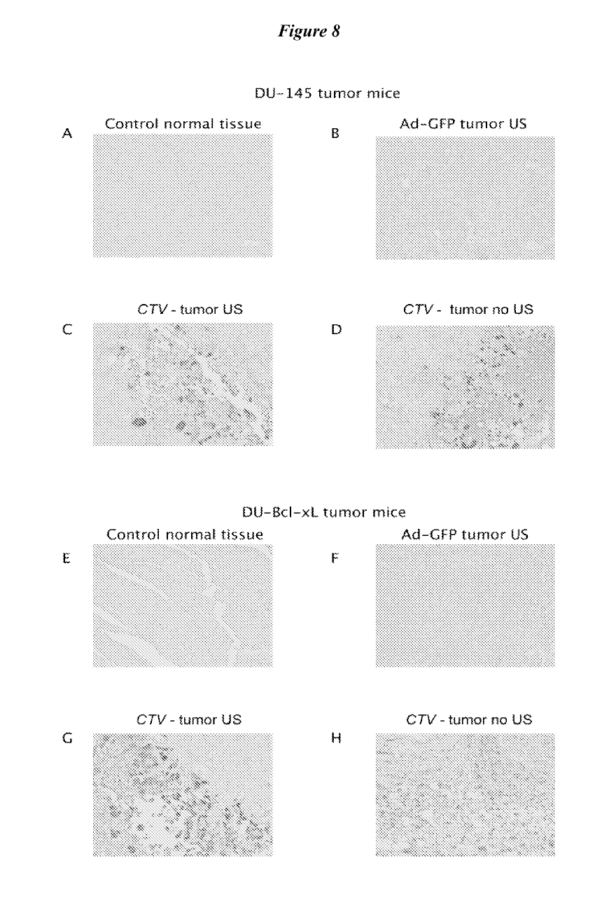

As previously observed with direct intratumoral injection, Ad.mda-7-microbubble complexes plus US was ineffective in reducing the growth of therapy resistant DU-Bcl-xL tumor xenografts (FIGS. 6C and 6D) [11]. In contrast, treatments with CTV-microbubble complexes plus US disrupted the tumor cyto-architecture, which correlated with an increase in the number of TUNEL positive tumor cells in both sonoporated right (FIG. 7E) and untreated left tumors (FIG. 7F). Control tumors treated with Ad-GFP-microbubble complexes were TUNEL negative, whereas tumors treated with Ad.mda-7-microbubble complexes showed small numbers of TUNEL positive cells FIG. 7C (sonoporated tumor on the right flank) and FIG. 7D (non-sonoporated tumor). As previously shown using intratumoral injection [11] the CTV virus when delivered by the microbubble plus US replicated in the treated tumor tissue as reflected by positive staining using antibody reacting with adenovirus E1A protein (FIG. 8). Additionally, as seen using the CTV and intratumoral injection of tumors on one flank of the animal, positive staining against E1A protein was found not only in the sonoporated tumor on the right flank but also on the non-sonoporated tumor on the left flank. Negative staining was found in the control tissues, GFP-transduced and normal tissues from a CTV transduced and US treated mouse. The observation that microbubble Ad-mediated gene therapy of CTV-microbubble complexes plus US completely eradicated the primary and the distant tumor (comparable to a metastasis) provides confidence that this strategy may prove amenable for successfully treating aggressive cancers for which intratumoral direct injection of the CTV virus is not possible due to location, number of metastases and spread of the malignancy to distant organ sites.

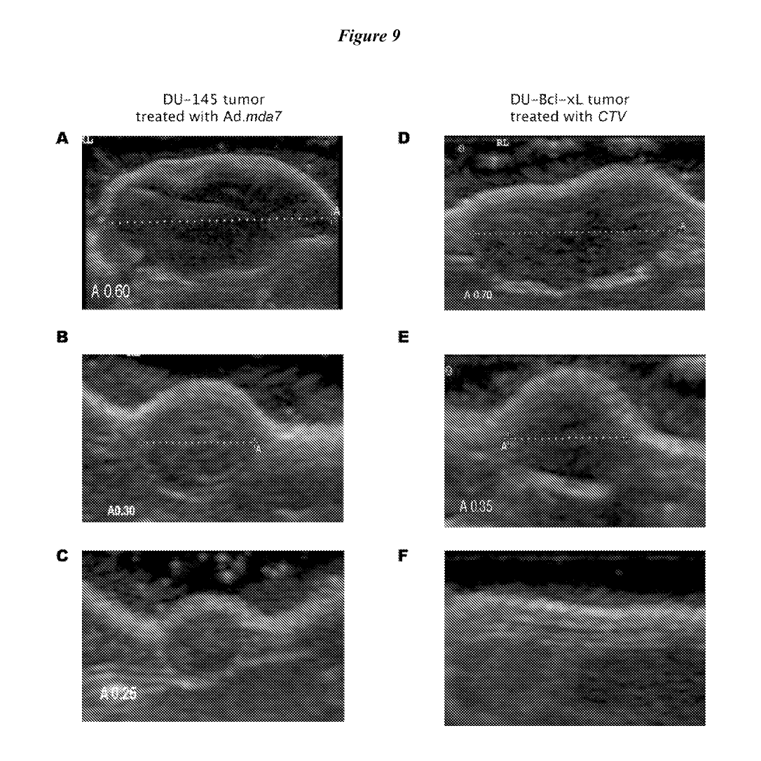

In this example, the size of the tumor was measured twice a week by caliper as well as by B-mode ultrasound scanning FIG. 9A shows the ultrasound image and measurements of a DU-145 tumor before treatment with Ad.mda-7-microbubble complexes. Panels B and C demonstrate the volume reduction in the same tumor after 2 and 4 weeks of treatments with Ad.mda-7-microbubble complexes and US. FIG. 9D shows the B-mode scan image and measurements of a DU-Bcl-xL tumor before treatment with CTV-microbubble complexes. Panels E and F emphasize the dramatic volume reduction in the same tumor after 2 and 4 wks of treatments with CTV-microbubble complexes and US leading to the eradication of the tumor xenograft. Additionally, no tumor regrowth in the primary or distant sites was evident CTV-microbubble complex and US-treated DU-Bcl-xL animals after an additional three weeks post-treatment. To investigate if the tumor would reappear after a longer period of time following the last treatment, three out of ten animals initially treated with CTV-microbubble complexes were not sacrificed at the endpoint of the study and were maintained for an additional 3 months. The mice were then sacrificed and dissected to look for potential tumor recurrence and/or eventual tumor spread. We did not observe any local tumor reappearance or distant metastasis in the lungs or liver in these mice that were treated with CTV-microbubble complexes and US indicating that this therapeutic approach could be suitable to target conditionally replication-competent adenoviruses (CRCA) to prostate tumors causing the eradication of localized as well as distant metastatic tumors.

Microbubbles have been used to protect viruses from rapid degradation by the immune system, thus allowing intravenous injection rather than direct target organ delivery by catheter-based approaches or operative bed injection [38]. However, variable levels of non-targeted gene expression have been noted in other organs such as the liver and lungs [38]. We have recently shown that US imaging and US contrast agents can increase target specificity of Ads to tumors, achieving transient transgene expression with strict image-guided site specificity by selecting microbubbles which completely enclosed the Ads in their gas filled core [12]. In our prior experience, US-mediated microbubble destruction improved the efficacy and reduced the non-specific expression of gene therapy vectors providing a useful tool for manipulating gene expression in the living animal.

Genetic therapies for prostate cancer represent promising strategies for the treatment of this neoplasm. The prostate gland is accessible by US, and potential therapeutic genes can be directed to this organ using portable diagnostic US platforms such as the SonoSite MicroMaxx (SonoSite, Inc., Bothell, Wash.) after a simple intravenous injection. Importantly, because prostate cancer is commonly a relatively slow-growing disease, it may be necessary to use repeated gene therapy applications, with single or multiple genes, over the life span of the patient. In these contexts, gene therapy protocols that delimit virus exposure to the immune system and can be administered multiple times during a patient's lifetime are appealing. This possibility will need to be explored in the future using tumor-bearing immune competent animals. In the current work, we explored the ability of US-mediated microbubble destruction to specifically deliver in prostate adenocarcinoma xenografts the mda-7/IL-24 gene [39, 40], that has been successfully employed in a Phase I clinical trial in patients with advanced solid tumors [6-9, 21-23, 28].

Potentially useful approaches for treating prostate and other cancers involve the use of a replication incompetent adenovirus (Ad.mda-7) or a conditionally replication competent Ad (Ad.PEG-E1A-mda-7; CTV) to administer the therapeutic cytokine mda-7/IL-24 to induce targeted therapy of tumors [6-9, 11, 33, 34, 37, 41]. Although very effective in prostate cancer cell lines, no therapeutic benefit is observed with Ad.mda-7 in the context of prostate cancer cells displaying elevated expression of Bcl-2 and/or Bcl-x_{L [}4, 11]. In contrast, administration of the CTV by direct intratumoral delivery in nude mice containing xenografted Bcl-x_{L} overexpressing DU-145 cells implanted on both flanks of the animal results in tumor eradication in both the primary injected tumor and the distant untreated tumor [11]. Conditionally replication competent Ads (CRCA), which induce oncolysis by cancer-specific replication, have been evaluated in several prostate cancer clinical trials [42, 43]. Most currently employed CRCA are based on the ONYX-015 backbone, which is dependent on the p53 status of the cancer cells and have shown only minimal objective clinical responses, thus limiting their universal applicability for the treatment of prostate or other cancers [44]. To this end, the novel CTV CRCA that employs the progression elevated gene-3 (PEG-3) promoter that functions in all types of cancer cells [11, 34, 37, 45, 46], irrespective of their p53 or Rb/retinoblastoma gene status, with very limited to no activity in normal cells has been constructed. In the cancer terminator virus (CTV), Ad replication through the E1A gene is driven by the cancer-specific promoter of progression elevated gene-3 (PEG-3) [47], which results in concomitant production of mda-7/IL-24 from the E3 region of the Ad. This CTV generates large quantities of MDA-7/IL-24 as a function of Ad replication uniquely in cancer cells that not only has cancer-selective apoptosis-inducing properties but also displays a plethora of indirect antitumor “bystander” activities, including distant tumor growth suppression and apoptosis, immune modulation and anti-angiogenesis [6-9, 22, 32, 35, 37, 41].

A limiting factor in effective gene therapy when employing intravenous viral delivery and when using CRCA is the effect of the immune system in neutralizing Ads [13]. In this context, a means of shielding the initial viral delivery vector using microbubbles in principle permits enhanced delivery of the viral payload to tumors when coupled with US [12]. In this example we have employed this strategy using CTV-microbubble complexes coupled with US to treat both DU-145 and therapy resistant DU-Bcl-xL established tumor xenografts on both flanks in nude mice. Systemic administration of the CTV-microbubble with US on the established right-sided tumor resulted in robust transgene expression and apoptosis induction with complete eradication of both the injected right side primary and distant (opposite flank; potentially representative of metastasis) human prostate cancers. However, no tumor regression was observed instead in mice bearing DU-145 and DU-Bcl-xL control tumors when injected intravenously with unprotected Ad.mda-7 and CTV viruses (see Supplemental figures s1 and s2), indicating that comparable doses of untargeted, unprotected viruses injected directly i.v. failed to elicit an antitumoral response. An exciting finding was that this protocol resulted in the indication of an enduring response in which no tumor regrowth occurred 3 months after cessation of the therapy protocol in the treated or untreated tumor site and additionally these animals had no signs of metastatic spread to the lungs or liver. Previous studies have indicated that the CTV when injected intratumorally will enter into the circulation, replicate and generate MDA-7/IL-24 protein in the primary and distant tumors in the nude mouse, predicting induction of a potential immune response [11, 34, 37, 41, 45].

Obvious questions are why mda-7/IL-24 serves as such an effective anti-tumor agent and why the CTV is superior to Ad.mda-7 as a viral-based therapeutic for primary and disseminated cancers? A noteworthy reason for the robustness of mda-7/IL-24 is the ability of this secreted cytokine to elicit a potent ‘bystander” anti-cancer effect [6]. mda-7/IL-24 can directly induce apoptosis when expressed inside cancer cells and can also induce growth suppression, apoptosis and endogenous MDA-7/IL-24 protein expression and secretion when added as a purified protein through interactions with the IL-20R1/IL-2082 and IL-22R1/IL-20R2 cell surface receptors [6, 8, 32, 35]. As a secreted cytokine, MDA-7/IL-24 also induces an array of potent immunomodulatory proteins from immune cells, including IL-6, IFN-γ, tumor necrosis factor-α, IL-1β, IL-12, and granulocyte macrophage colony-stimulating factor [32]. These cytokines secreted by peripheral blood mononuclear cells can activate antigen-presenting cells to present tumor antigens, thereby triggering an antitumor immune response [48]. These observations have been recapitulated in a Phase I clinical trial involving intratumoral injection of Ad.mda-7 (INGN 241) in patients with advanced carcinomas and melanomas [8, 21, 28]. In principle, the ‘bystander” effects elicited by MDA-7/IL-24 are concentration dependent [35] and large amounts of this cytokine generated by the CTV would be predicted to have an enhanced therapeutic impact in the patient. Moreover, the immunomodulatory functions of mda-7/IL-24 would be particularly significant in a patient with an intact immune system where the generation of robust amounts of mda-7/IL-24 by the CTV might result in an amplified immune response against the cancer cells. The potent activity of the CTV compared to Ad.mda-7 suggests a need for only limited administration of this CRCA [11, 34, 37, 41], which would work extremely well in the context of microbubble-Ad complexes. In principle, the microbubble approach would further minimize the activation of the immune system against the Ad that would normally promote viral clearance. We presently confirm for the first time that microbubble-assisted delivery of the CTV can serve as a valuable therapeutic tool to combat therapy resistant prostate cancer. As previously emphasized, the CTV-microbubble injected US treated mice appeared to be disease free 3 months after therapy cessation suggesting that a cure was established.

In summary, this example support the proposition that US-directed delivery of CTV-microbubble complexes might provide a nontoxic and effective alternative or complement to conventional adjuvant treatment modalities for patients with primary and metastatic prostate cancer. Based on previous studies of combinatorial therapy preclinical-trials in cell culture and in animal models, a combination of CTV-microbubble approach employing US with localized low-dose radiotherapy might promote an even more profound effect, potentially further enhancing mice survival [9, 49].

Cell lines, cell culture and adenoviral production. The DU-145 (human prostate adenocarcinoma), cell line was obtained from the American Type Culture Collection (ATCC, Rockville, Md.) and the DU-Bcl-xL cell line, which constitutively expresses elevated levels of Bcl-x_{L} has been described previously [4]. The cell lines were grown at 37° C., in a 5% CO2/95% atmosphere, in Dulbecco's modified Eagle's medium (Mediatech Inc., Herndon, Va.) supplemented with 10% fetal bovine serum (FBS) from Hyclone, Inc., (Logan, Utah). Ad-GFP, which expresses the green fluorescence protein gene under the strong cytomegalovirus (CMV) constitutive promoter was generated using the AdEasy system (Carlsbad, Calif.); the conditionally replication competent cancer terminator virus CTV (Ad.PEG-E1A-mda-7) [11, 34, 37] and Ad.mda-7 [26] were amplified and purified with the BD Adeno-X virus purification kit (BD Biosciences, Mountain View, Calif.) following manufacturer's directions. Viral titers were determined by a plaque assay and the titer was adjusted to 1.2×10¹² plaque-forming units (pfu)/mL as described [26].

Preparation of microbubbles and US platform. Targeson (Targeson, Inc. San Diego, Calif.) custom synthesis US contrast agent (perfluorocarbon microbubbles, encapsulated by a lipid monolayer and poly(ethyleneglycol) stabilizer) were prepared following manufacturer's instructions. Microbubbles were reconstituted in the presence or absence of 1 mL of 1.2×10¹² pfu of Ads and unenclosed, surface associated Ads were inactivated as previously described [12]. For in vivo experiments US exposure was achieved with a Micro-Maxx SonoSite (SonoSite, Bothell, Wash.) US machine equipped with the transducer L25 set at 0.7 Mechanical Index (MI), 1.8 MPa for 10 min.

Antibodies and Western Blot analysis. DU-145 cells were transduced with 50 MOI of Ad.mda-7 or Ad-CMV as a control and 24 or 48-hr post transduction 50 μg of total cell extracts were subjected to Western blot analysis using a mouse monoclonal anti-MDA-7/IL-24 (GenHunter, Inc, Nashville, Tenn.) (1:2,000 incubation for 1 hr) or the mouse monoclonal anti-GAPDH sc-0411 (1:5,000 incubation for 1 hr) (Santa Cruz, Santa Cruz, Calif.), as control. Western blot analysis was also conducted on protein extracts from microbubble/US assisted in vivo transfer of Ad-GFP or mda-7/IL-24 using antibodies that specifically recognized GFP sc-53882 (SantaCruz, Santa Cruz, Calif.), MDA-7/IL-24 (GenHunter, Nashville, Tenn.), and β-actin sc-47778 (SantaCruz, Santa Cruz, Calif.). Briefly, 96 hr following targeted microbubble/US assisted in vivo transfer of Ad-GFP, mice were sacrificed and fresh tumor (right and left flank), heart, lung, liver, and kidney tissues were harvested and snap frozen in liquid nitrogen. Mice receiving mda-7/IL-24 gene-microbubble US guided therapy were sacrificed at the endpoint of the study (5-6 wks after gene therapy injections). Tissues were homogenized and equal amounts of proteins were run on a SDS-PAGE and transferred to a nitrocellulose membrane. The membrane was then incubated with the monoclonal anti-GFP 1:2,000 for 1 hr at room temperature and then washed three times in TBS-T. Monoclonal anti-MDA-7/IL-24 (GenHunter, Inc, Nashville, Tenn.) was incubated 1:2,000 for 1 hr at room temperature and then washed three times in TBS-T. Monoclonal anti β-actin (1:5,000) was incubated 1 hr at room temperature and then washed three times in TBS-T. Appropriate secondary HRP-conjugated antibodies 1:20,000 were incubated 45 min at room temperature and washed three times with TBS-T. Signals were developed on an X-ray film after reaction with an Electrogenerated ChemiLuminescence (ECL) Supersignal kit (Pierce, Rockford, Ill.).

Animal study and ultrasonic bubble destruction. Animal studies were performed in accordance with NIH recommendations and the approval of the institutional animal research committee. Animal care and humane use and treatment of mice were in strict compliance with (1) institutional guidelines, (2) the Guide for the Care and Use of Laboratory Animals (National Academy of Sciences, Washington, D.C., 1996), and (3) the Association for Assessment and Accreditation of Laboratory Animal Care International (Rockville, Md., 1997). All the animals used in these studies were 8- to 12-week-old female/male congenitally athymic BALB/c nude mice, homozygous for the nu/nu allele, bred in our laboratory. The colony of the mice was developed from breeding stock obtained from Charles Rivers Laboratories, Wilmington, Mass. The mice were maintained in isolation in autoclaved cages with polyester fiber filter covers, under germ-free conditions; all food, water, and bedding were sterilized. A total of about 420 nude mice (n=10 each experimental point) were implanted with the human prostate adenocarcinoma cell lines (DU-145 or DU-Bcl-xL) as a xenograft model (injecting 1.5×10⁶ or 2.5×10⁶ cancer cells on each flank of the animal). After ˜30-days, mice were sedated in an IMPAQ6 anesthesia apparatus (VetEquip Inc, Pleasanton, Calif.) that was saturated with 3-5% Isofluorane and 10-15% oxygen with the aid of a precision vaporizer (VetEquip Inc, Pleasanton, Calif.) to deliver the appropriate amount of anesthetic and to induce anesthesia. The mice were placed on a warmed mat with 37° C. circulating water for the entire procedure. A27-gauge needle with a heparin lock was placed within a lateral tail vein for administration of contrast material. The nude mice received injections of 100 μL of microbubbles with/without Ads through the tail vein for 5 wks/once a wk. The mice were split into two control groups (one control group receiving 100 μL of microbubbles and US, and another control group receiving both microbubbles/Ad-GFP and US) and eight active groups of 10 mice each (all receiving microbubbles and Ad.mda-7 or CTV and US). Six additional control groups were set up which received direct i.v. injections of 100 μL of the Ads (Ad-GFP, Ad.mda7/IL-24, or CTV) in the presence or not of US. Grayscale US imaging was performed with a SonoSite scanner (SonoSite, Bothell, Wash.) equipped with the transducer L25 set at 0.7 Mechanical Index (MI), 1.8 MPa for 10 min. Ultrasound images were recorded as digital clips. In every experiment, 10 animals for each treatment or control group were used to study tumor regression. Every experiment was repeated at least twice. Tumor volumes were determined by measuring the tumors twice a wk with either a caliper or by ultrasound measurements of the tumor axes. Tumor volumes were determined using the following formula: V=(π/8)a×b², where V is the tumor volume, a is the maximum tumor diameter, and b is the diameter at 90° to a [50]. The mice were humanely sacrificed by placing them in a CO₂ gas jar placed in a ventilated fume hood. The tumors (right and left flank), heart, lungs, kidneys, and liver were harvested. Tissues to be sectioned were dry snap frozen or placed either in OCT (Sakura Finetek USA, Inc., Torrance, Calif.), frozen in liquid nitrogen, and stored at −80° C. or were preserved in neutral buffered formalin at 4° C. prior to embedding in paraffin for immunohistochemical analysis.

Determination of apoptotic cells by TUNEL assay. A TUNEL method was used for the detection of apoptotic cells. For this purpose, we used the DeadEnd Colorimetric TUNEL Assay kit (Promega, Madison, Wis.) according to the manufacturer's instructions. Briefly, paraffin-embedded slides were deparaffinized and rehydrated. Pre-equilibrated slides were labeled with a labeling DNA-strand breaks solution containing a biotinylated nucleotide mix (60 minutes at 37° C.). After several washes in 2×SSC and PBS, slides were blocked with hydrogen peroxide (3-5 min at room temperature). After several washes in PBS, the slides were incubated with streptavidin-HRP conjugated diluted in PBS (30 min at room temperature). DAB was used as the final chromogen and Hematoxylin was used as a counterstaining procedure. Apoptotic cells on the slides were observed under an Olympus light microscope (400× magnification) in randomly chosen fields.

Statistical Analysis. All statistical analyses were performed by using SAS version 9.1. Comparisons of tumor volumes were done separately three times: before the treatment, two weeks after the treatment, and at the end of the study. Statistical analyses for comparisons of different types of treatments were done using one-way ANOVA followed by Tukey-Kramer multiple adjusted pair wise tests. P-value <0.05 was considered significant.

### REFERENCES

1. Damber, J E, and Aus, G (2008). Prostate cancer. Lancet 371: 1710-1721.
2. Moon, C, Park, J C, Chae, Y K, Yun, J H, and Kim, S (2008). Current status of experimental therapeutics for prostate cancer. Cancer Lett 266: 116-134.
3. Shi, X B, Gumerlock, P H, and deVere White, R W (1996). Molecular biology of prostate cancer. World J Urol 14: 318-328.
4. Lebedeva, I V, et al. (2003). Bcl-2 and Bcl-x(L) differentially protect human prostate cancer cells from induction of apoptosis by melanoma differentiation associated gene-7, mda-7/IL-24. Oncogene 22: 8758-8773.
5. Lebedeva, I V, et al. (2003). Melanoma differentiation associated gene-7, mda-7/interleukin-24, induces apoptosis in prostate cancer cells by promoting mitochondrial dysfunction and inducing reactive oxygen species. Cancer Res 63: 8138-8144.
6. Fisher, P B (2005). Is mda-7/IL-24 a “magic bullet” for cancer? Cancer Res 65: 10128-10138.
7. Fisher, P B, et al. (2003). mda-7/IL-24, a novel cancer selective apoptosis inducing cytokine gene: from the laboratory into the clinic. Cancer Biol Ther 2: S23-37.
8. Lebedeva, I V, et al. (2007). mda-7/IL-24, novel anticancer cytokine focus on bystander antitumor, radiosensitization and antiangiogenic properties and overview of the phase I clinical experience (Review). Int J Oncol 31: 985-1007.
9. Lebedeva, I V, et al. (2005). mda-7/IL-24: exploiting cancer's Achilles' heel. Mol Ther 11: 4-18.
10. Sarkar, D, Dent, P, Curiel, D T, and Fisher, P B (2008). Acquired and innate resistance to the cancer-specific apoptosis-inducing cytokine, mda-7/IL-24: not insurmountable therapeutic problems. Cancer Biol Ther 7: 109-112.
11. Sarkar, D, et al. (2007). Eradication of therapy-resistant human prostate tumors using a cancer terminator virus. Cancer Res 67: 5434-5442.
12. Howard, C M, Forsberg, F, Minimo, C, Liu, J B, Merton, D A, and Claudio, P P (2006). Ultrasound guided site-specific gene delivery system using adenoviral vectors and commercial ultrasound contrast agents. J Cell Physiol 209: 413-421.
13. Jiang, H, Wang, Z, Serra, D, Frank, M M, and Amalfitano, A (2004). Recombinant adenovirus vectors activate the alternative complement pathway, leading to the binding of human complement protein C3 independent of anti-ad antibodies. Mol Ther 10: 1140-1142.
14. Lawrie, A, Brisken, A F, Francis, S E, Cumberland, D C, Crossman, D C, and Newman, C M (2000). Microbubble-enhanced ultrasound for vascular gene delivery. Gene Ther 7: 2023-2027.
15. Ng, K Y, and Liu, Y (2002). Therapeutic ultrasound: its application in drug delivery. Med Res Rev 22: 204-223.
16. Larina, I V, Evers, B M, and Esenaliev, R O (2005). Optimal drug and gene delivery in cancer cells by ultrasound-induced cavitation. Anticancer Res 25: 149-156.
17. Howard, C M (2004). The role of ultrasound contrast agents in gene therapy. Applied Radiology 33, suppl: 126-135.
18. Goldberg, B B, Liu, J B, and Forsberg, F (1994). Ultrasound contrast agents: a review. Ultrasound Med Biol 20: 319-333.
19. Pitt, W G, Husseini, G A, and Staples, B J (2004). Ultrasonic drug delivery—a general review. Expert Opin Drug Deliv 1: 37-56.
20. Dayton, P A, and Rychak, J J (2007). Molecular ultrasound imaging using microbubble contrast agents. Front Biosci 12: 5124-5142.
21. Cunningham, C C, et al. (2005). Clinical and local biological effects of an intratumoral injection of mda-7 (IL24; INGN 241) in patients with advanced carcinoma: a phase I study. Mol Ther 11: 149-159.
22. Eager, R, Harle, L, and Nemunaitis, J (2008). Ad-MDA-7; INGN 241: a review of preclinical and clinical experience. Expert Opin Biol Ther 8: 1633-1643.
23. Fisher, P B, et al. (2007). Melanoma differentiation associated gene-7/interleukin-24 (mda-7/IL-24): novel gene therapeutic for metastatic melanoma. Toxicol Appl Pharmacol 224: 300-307.
24. Inoue, S, et al. (2007). mda-7 in combination with bevacizumab treatment produces a synergistic and complete inhibitory effect on lung tumor xenograft. Mol Ther 15: 287-294.
25. Ramesh, R, et al. (2004). Local and systemic inhibition of lung tumor growth after nanoparticle-mediated mda-7/IL-24 gene delivery. DNA Cell Biol 23: 850-857.
26. Su, Z Z, et al. (1998). The cancer growth suppressor gene mda-7 selectively induces apoptosis in human breast cancer cells and inhibits tumor growth in nude mice. Proc Natl Acad Sci USA 95: 14400-14405.
27. Tahara, I, et al. (2007). Systemic cancer gene therapy using adeno-associated virus type 1 vector expressing MDA-7/IL24. Mol Ther 15: 1805-1811.
28. Tong, A W, et al. (2005). Intratumoral injection of INGN 241, a nonreplicating adenovector expressing the melanoma-differentiation associated gene-7 (mda-7/IL24): biologic outcome in advanced cancer patients. Mol Ther 11: 160-172.
29. Yacoub, A, et al. (2003). MDA-7 (interleukin-24) inhibits the proliferation of renal carcinoma cells and interacts with free radicals to promote cell death and loss of reproductive capacity. Mol Cancer Ther 2: 623-632.
30. Zerbini, L F, et al. (2006). A novel pathway involving melanoma differentiation associated gene-7/interleukin-24 mediates nonsteroidal anti-inflammatory drug-induced apoptosis and growth arrest of cancer cells. Cancer Res 66: 11922-11931.
31. Nishikawa, T, Ramesh, R, Munshi, A, Chada, S, and Meyn, R E (2004). Adenovirus-mediated mda-7 (IL24) gene therapy suppresses angiogenesis and sensitizes NSCLC xenograft tumors to radiation. Mol Ther 9: 818-828.
32. Gupta, P, et al. (2006). mda-7/IL-24: multifunctional cancer-specific apoptosis-inducing cytokine Pharmacol Ther 111: 596-628.
33. Sarkar, D, et al. (2007). Melanoma differentiation associated gene-7 (mda-7)/IL-24: a ‘magic bullet’ for cancer therapy? Expert Opin Biol Ther 7: 577-586.
34. Sarkar, D, Su, Z Z, Vozhilla, N, Park, E S, Gupta, P, and Fisher, P B (2005). Dual cancer-specific targeting strategy cures primary and distant breast carcinomas in nude mice. Proc Natl Acad Sci USA 102: 14034-14039.
35. Sauane, M, et al. (2008). Autocrine regulation of mda-7/IL-24 mediates cancer-specific apoptosis. Proc Natl Acad Sci USA 105: 9763-9768.
36. Su, Z, et al. (2005). Unique aspects of mda-7/IL-24 antitumor bystander activity: establishing a role for secretion of MDA-7/IL-24 protein by normal cells. Oncogene 24: 7552-7566.
37. Sarkar, D, et al. (2008). A cancer terminator virus eradicates both primary and distant human melanomas. Cancer Gene Ther 15: 293-302.
38. Beeri, R, Guerrero, J L, Supple, G, Sullivan, S, Levine, R A, and Hajjar, R J (2002). New efficient catheter-based system for myocardial gene delivery. Circulation 106: 1756-1759.
39. Jiang, H, Lin, J J, Su, Z Z, Goldstein, N I, and Fisher, P B (1995). Subtraction hybridization identifies a novel melanoma differentiation associated gene, mda-7, modulated during human melanoma differentiation, growth and progression. Oncogene 11: 2477-2486.
40. Jiang, H, Su, Z Z, Lin, J J, Goldstein, N I, Young, C S, and Fisher, P B (1996). The melanoma differentiation associated gene mda-7 suppresses cancer cell growth. Proc Natl Acad Sci USA 93: 9160-9165.
41. Sarkar, D, Su, Z Z, and Fisher, P B (2006). Unique conditionally replication competent bipartite adenoviruses-cancer terminator viruses (CTV): efficacious reagents for cancer gene therapy. Cell Cycle 5: 1531-1536.
42. Freytag, S O, et al. (2003). Phase I study of replication-competent adenovirus-mediated double-suicide gene therapy in combination with conventional-dose three-dimensional conformal radiation therapy for the treatment of newly diagnosed, intermediate- to high-risk prostate cancer. Cancer Res 63: 7497-7506.
43. Small, E J, et al. (2006). A phase I trial of intravenous CG7870, a replication-selective, prostate-specific antigen-targeted oncolytic adenovirus, for the treatment of hormone-refractory, metastatic prostate cancer. Mol Ther 14: 107-117.
44. Heise, C, Sampson-Johannes, A, Williams, A, McCormick, F, Von Hoff, D D, and Kirn, D H (1997). ONYX-015, an E1B gene-attenuated adenovirus, causes tumor-specific cytolysis and antitumoral efficacy that can be augmented by standard chemotherapeutic agents. Nat Med 3: 639-645.
45. Sarkar, D, et al. (2005). Targeted virus replication plus immunotherapy eradicates primary and distant pancreatic tumors in nude mice. Cancer Res 65: 9056-9063.
46. Su, Z Z, et al. (2005). Targeting gene expression selectively in cancer cells by using the progression-elevated gene-3 promoter. Proc Natl Acad Sci USA 102: 1059-1064.
47. Su, Z Z, Shi, Y, and Fisher, P B (1997). Subtraction hybridization identifies a transformation progression-associated gene PEG-3 with sequence homology to a growth arrest and DNA damage-inducible gene. Proc Natl Acad Sci USA 94: 9125-9130.
48. Gao, P, et al. (2008). Secretable chaperone Grp170 enhances therapeutic activity of a novel tumor suppressor, mda-7/IL-24. Cancer Res 68: 3890-3898.
49. Yacoub, A, et al. (2008). MDA-7/IL-24 plus radiation enhance survival in animals with intracranial primary human GBM tumors. Cancer Biol Ther 7: 917-933.
50. Puisieux, I, et al. (1998). Canarypox virus-mediated interleukin 12 gene transfer into murine mammary adenocarcinoma induces tumor suppression and long-term antitumoral immunity. Hum Gene Ther 9: 2481-2492.

### Example 2

Melanoma differentiation associated gene-7 (mda-7), also known as interleukin-24 (IL-24) was initially cloned by subtraction hybridization applied to a ‘differentiation therapy’ model of human melanoma cancer (1). Subsequent studies confirmed an inverse correlation between mda-7/IL-24 expression and melanoma development and progression (2). A large body of evidence has demonstrated that gene delivery of mda-7/IL-24 by means of a plasmid or via a replication incompetent adenovirus (Ad.mda-7) promotes growth suppression and induces apoptosis in a broad array of human cancers. In contrast, mda-7/IL-24 does not induce growth suppressive or toxic effects in normal cells (3-5). In addition to induction of cancer-specific apoptosis, mda-7/IL-24 is also capable of regulating cell cycle (6), inhibiting angiogenesis (7), sensitizing cancer cells to radiation therapy (8, 9). Recent studies highlighted a potent ‘bystander’ antitumor activity exhibited by mda-7/IL-24 in adjacent tumor cells (10), which may provide a means of obviating the technical difficulty of transducing the entire tumor mass with this gene. Although the mechanism of cancer cell selectivity of mda-7/IL-24 requires further delineation (11-13), these pleiotropic properties places mda-7/IL-24 in a unique position for a novel cancer gene therapy. Indeed, a replication-incompetent adenovirus expressing mda-7/IL-24 (INGN-241) has now undergone evaluation in a Phase I clinical trial for multiple solid tumors, and has demonstrated safety and significant clinical activity (5). The fact that this targeted therapy results in tumor cell death and does not affect normal cells provides further hope that its efficacy will not be accompanied by side effects seen with many other therapies.

Prostate cancer (CaP) is the most prevalent cancer and the second leading cause of cancer-related death in American men. Currently, there is no curative therapy for patients who develop recurrences or for those who have metastatic disease at the time of diagnosis. A systemic, specific and sustained immune response against cancer at the time of initial therapy could address the most critical issue: prevention of tumor recurrence. However, CaP is considered poorly immunogenic despite the presence of antigens that may be tumor specific (14). The antigenic peptide repertoires derived from the random genetic mutations and tumor-associated antigens potentially can induce individual tumor-specific immunity. In most cases, simply killing tumor cells by molecular or tumor-targeted treatments including mda-7/IL-24 gene therapy may not be sufficient to raise effective antitumor immunity. Without a proper ‘danger’ signals, the apoptotic cells are largely ignored by the immune system (15), or may even induce tolerance (16). Only ‘inflammatory’ or ‘immunogenic’ cell killing, which is distinguished from normal homeostatic processes, can be sensed by the host immune system and is able to trigger the activation of immune components (17-19).

In recent years, some stress proteins have gained widespread attention due to their potential roles in cancer immunotherapy. The antitumor response has largely been attributed to the ability of some stress proteins to form complexes with tumor-derived antigens and thereby facilitate the antigen cross-presentation and priming of T-effector cells. Different stress proteins are highly different in cellular functions and in their abilities to chaperone or bind antigens. Grp170, the newly characterized stress protein and the largest endoplasmic reticulum (ER)-resident molecular chaperone, is a highly “diverged” relative of the hsp70 family (20). Grp170 has been shown to interact with transporter associated with antigen processing (TAP) translocated peptides and may be involved in polypeptide trafficking in the antigen presentation pathway (21, 22). Our previous studies have demonstrated grp170 is capable of interacting with specific receptors on professional antigen-presenting cells (APCs) and shuttling antigens into the endogenous presentation pathway efficiently, which results in antigen presentation and tumor-specific immunity (23, 24). Tumor-derived grp170 or grp170 complexed with tumor-associated antigens are highly effective in eliciting potent antigen and tumor specific antitumor immune responses in various murine tumor models (23, 25). In addition to promoting antigen cross-presentation, grp170 also acts as a ‘danger’ signal that stimulates phenotypic and functional maturation of DCs, as indicated by up-regulation of MHC class II and co-stimulatory molecules, secretion of proinflammatory cytokines and chemokines (26). More recently, we demonstrated that extracellular targeting of grp170 by molecular engineering strongly enhanced the immunogenicity of a poorly immunogenic tumor in vivo (27).

In view of multiple undefined antigens present endogenously within prostate cancer cells, it is conceivable that concurrent delivery of a potent immunostimulatory agent while inducing tumor cell death in situ may lead to a potent antitumor immunity capable of eliminating both local and distant tumors. The ability of grp170 to simultaneously deliver both adjuvanticity for the activation of innate immunity and antigenicity for CTL cross-priming could dramatically increase the intrinsic immunogenicity of the in vivo tumor killing by mda-7/IL-24. In this study, we sought to examine the potential of grp170-based immunotherapy to enhance mda-7/IL-24-mediated prostate cancer therapy. To exploit the adjuvant-like properties of the protein and its ability to bind and present tumor-derived antigens to APCs, we engineered a replication incompetent adenovirus that can efficiently infect prostate tumor cells and mediate the expression of a modified, secretable grp170 gene. We show that intratumoral co-administration of secretable grp170 and mda-7/IL-24 effectively and markedly suppress treated local prostate tumors, resulting in complete tumor regression in some cases. Furthermore, the combined therapies that target both tumor and immune system augment a robust tumor-specific immune response, leading to a significantly improved control of distant and secondary tumors.

Mice and Cell lines. 8 to 12-week-old male C57BL/6 mice purchased from the National Institutes of Health animal facilities were maintained in a pathogen-free facility at Roswell Park Cancer Institute. Animal care and experiments were approved by the Institutional Animal Care and Use Committee. TRAMP-C2 cell line was derived from a prostate tumor that arose in a TRAMP (Transgenic Adenocarcinoma of Mouse Prostate) mouse in the C57BL/6 background (28). The TRAMP-C2 cells, C2 cells transduced with OVA (C2-OVA) and B16 melanoma cells are maintained in DMEM containing 10% fetal bovine serum, 2 mM L-glutamine, and 100 U/ml penicillin/streptomycin.

Adenovirus construction and characterization. The recombinant replication-defective Ad.mda-7 virus was created in two steps as described previously (3). The adenovirus carrying a secretable form of the grp170 gene (Ad.sgrp170) was constructed using BD Adeno-X™ Adenoviral Expression System (BD Bioscience, Palo Alto, Calif.). To distinguish the secretable grp170 from endogenous grp170, a His-tag was fused to the C-terminus of mouse grp170, in which the KNDEL endoplasmic reticulum signal has been eliminated (27). This modified cDNA was inserted into the Nhe I/Xba I cloning sites of the pShuttle 2 plasmid, and subsequently cloned into I-Ceu I/PI-Sce I sites of Adenoviral vector. All adenoviral vectors were produced in HEK293 cells and infection titers were determined by plaque tittering on 293 cells. Viruses were concentrated and purified using AdenoPACK Maxi columns (Sartorius Stedim Biotech) according to the procedure provided by the manufacturer. Endotoxin levels are determined by using a chromogenic limulus amebocyte lysate kinetic assay kit (Kinetic-QCL; Biowhittaker, Walkersville, Md.). Cells were infected with viruses with different multiplicity of infection (MOI) under standard culture conditions. Expression of the secreted grp170 in the supernatants of the infected cells was examined using antibodies against grp170 and His-tag as previously described (27). Expression of mda-7 μL-24 was examined by immunoblotting using murine anti-mda-7/IL-24 monoclonal antibodies (Gene Hunter, Inc).

Apoptosis assays. Annexin V binding assays were used to determine apoptosis induction as previous described (29). Briefly, 24 h after virus infection at a MOI of 300 plaque-forming units (pfu) per cell, cells were harvested, washed and resuspended in binding buffer containing 2 mM CaCl₂. Cells were stained with FITC-labeled Annexin V (BD Biosciences, Palo Alto, Calif.) and propidium iodide for 15 min at room temperature, and analyzed by Flow cytometry. In addition, cells were subjected to immunoblotting analysis using antibodies for Poly (ADP-ribose) polymerase (PARP) (Santa Cruz Biotech).

Cell proliferation assay. Cells (2×10⁴ cells/well) were seeded in 96-well tissue culture plates and treated with Ad.mda-7 at a MOI of 300. At the indicated times, medium is removed, and 100 μl PBS containing 5 mg/ml MTT (Sigma, St. Louis, Mo.) is added to each well. The cells were incubated at 37° C. for 4 h and then an equal volume of solublization solution (0.01 N HCl in 10% SDS) is added to each well and mixed thoroughly. The absorbance from the plates is read on a Bio-Rad Microplate Reader at 595 nm.

Tumor studies. 2×10⁶ TRAMP-C2 tumor cells suspended in 100 μl sterile PBS were injected into the left dorsal flank of mice. When the tumors reach 4-5 mm² (approximately one week after tumor inoculation), animals were randomly divided into five groups and received Ad.GFP, Ad.mda-7, Ad.sgrp170, or Ad.mda-7 plus Ad.sgrp170. Viruses were administrated intratumorally in 50 μl PBS (5×10⁸ pfu per mouse). For the group receiving the combined therapies, 2.5×10⁸ pfu of each virus was administrated. All treatments were given every other day for a total of 4 doses. Tumor growth is monitored by measuring perpendicular tumor diameters using an electronic digital caliper. To determine the effect of the combined therapies on distant tumors, mice were established with tumors in both flanks The different adenoviruses as described above were delivered into tumors in the left flank only. Growth of contralateral tumors was followed to determine systemic antitumor immunity. Depletion of CD4⁺, CD8⁺ T-cell subsets was accomplished by i.p. injection of 200 μg GK1.5 and 2.43 mAb respectively as previously described (27). Effective depletion of cell subsets was maintained by the antibody injections once a week for the duration of experiment.

Enzyme-linked immunosorbent spot (ELISPOT) and CTL assays. Splenocytes were isolated from immunized mice two weeks after immunization and stimulated with 1 μg/ml H-2K^{b} restricted CTL epitope OVA₂₅₇₋₂₆₄ (SIINFEKL) or mitomycin C-treated C2 tumor cells to determine antigen-specific, IFN-γ secreting T-cells as previously described (30). For CTL assay, splenocytes were stimulated with mitomycin C-treated tumor cells or 1 μM OVA₂₅₇₋₂₆₄ in the presence of IL-2 (20 U/ml) for 6 days. CD8⁺ T-cells were used as effector cells in a chromium release assay as described (30).

Statistical analysis. Statistical analysis of tumor growth inhibition, cytotoxicity assays, and ELISPOT assay were done using paired or unpaired Student's t test, as appropriate. Ps<0.05 were considered statistically significant.

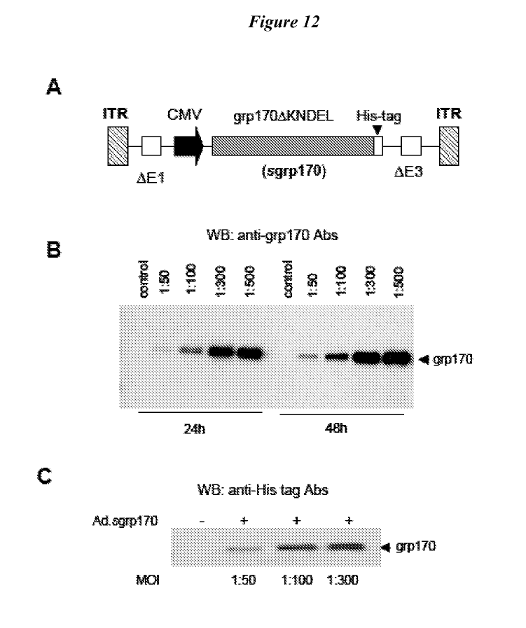

Construction and validation of adenovirus vector encoding secretable grp170. We recently reported that extracellular targeting of grp170 significantly improved the immunogenicity of a poorly immunogenic tumor (27). To enhance the immunogenicity of mda-7-mediated tumor cell-specific apoptosis and promote systemic antitumor immunity, we constructed a secreted form of mouse grp170 (sgrp170) by deleting the COOH-terminal ER-retention signal ‘KNDEL’ (FIG. 12A). Therefore, instead of being retained in the ER, the modified grp170 should be secreted after protein synthesis. To distinguish the modified gene product from the endogenous grp170, the His-tag was fused to the COOH terminus of the sgrp170 gene. The fusion gene of sgrp170-His was inserted into E1/E3-deleted adenovirus-based vectors under the control of CMV promoter for constitutive and effective gene expression (i.e., Ad.sgrp170). The replication-defective virus vectors encoding this new fusion gene were successfully packaged and expanded in HEK293 cells.

As a model for our studies, we selected the TRAMP-C2 cell line that was established from the spontaneous tumor of the autochthonous transgenic adenocarcinoma of mouse prostate (TRAMP) model (28). Upon subcutaneous transplantation into syngeneic male C57BL/6 mice, TRAMP-C2 cells form slowly growing, vascularized and poorly immunogenic tumors. Following infection of C2 tumor cells with Ad.sgrp170 at different MOIs, expression of the secretable grp170 gene was examined in the supernatants of the infected cells. Robust expression of the sgrp170 gene could be observed from day 1 at a MOI of 100 (FIG. 12B). An increase in the level of secreted grp170 was seen in cells infected with Ad.sgrp170 at a higher MOI (FIG. 12B). The expression peaked at around 48 h and remained stable for up to 4 d (data not shown). The secretable form of modified grp170 in the supernatants was further verified by immunoblotting using anti-His-tag antibodies (FIG. 12C). In addition, the infection of C2 cells with Ad.sgrp170 at a MOI of up to 500 had no observable effect on C2 cell viability in vitro (data not shown), indicating that sgrp170 overexpression per se does not exert a cytotoxic antitumor effect.

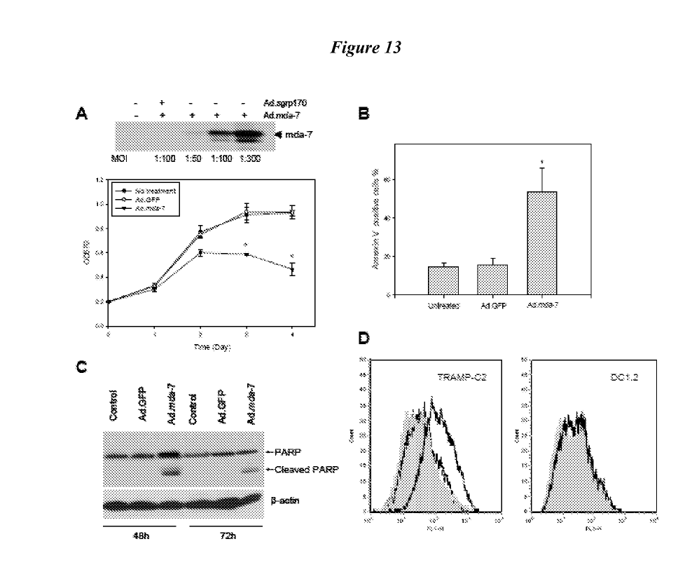

Adenovirus-mediated mda-7/IL-24 expression inhibits TRAMP-C2 tumor cell growth by inducing tumor apoptosis. In light of the limited information on the antitumor activity of mda-7/IL-24 in murine tumor cells, we therefore first determined whether adenovirus-mediated mda-7/IL-24 expression could induce growth suppression and apoptosis in TRAMP-C2 tumor cells (FIG. 13). Immunoblotting analysis confirmed the expression of mda-7/IL-24 gene in C2 cells infected with Ad.mda-7 at different MOIs (FIG. 13A, upper). Ad.mda-7 generated multiple bands because of glycosylation, ranging in size from 20 to 30 kDa. A significant inhibition of proliferation (p=0.001) was observed in C2 tumor cells treated with Ad.mda-7 at a MOI of 300 compared with that in cells treated with PBS or Ad.GFP (FIG. 13A, bottom). Treatment with Ad.mda-7 showed no significant growth inhibition in normal cells (data not shown), consistent with previous reports (5, 31). In addition, co-infection of C2 cells with Ad.sgrp170 had no effect on mda-7/IL-24-induced growth suppression in C2 tumor cells (data not shown).

Annexin V staining followed by fluorescent-activated cell sorting analysis (FACS) was carried out to determine early apoptotic changes in C2 cells after infection with 300 pfu/cell of Ad.GFP or Ad.mda-7 (FIG. 13B). Whereas significant increase in apoptotic cells were observed in C2 tumor cells following Ad.mda-7 infection, no such change was evident in C2 cells treated with Ad.GFP or left untreated. In addition, cleavage of PARP was detected only in C2 cells infected with Ad.mda-7 (FIG. 13C), suggesting that overexpression of mda-7/IL-24 in mouse tumor cells induces apoptosis in an manner similar to that observed in human cancer cells (31). Furthermore, the effect of Ad.mda-7 on the viability of cancer and normal cells was evaluated by Annexin V staining and FACS (FIG. 13D). The Ad.mda-7 infection resulted in a profound increase in apoptosis of C2 tumor cells. In contrast, no significant apoptosis was seen in normal cell line DC1.2 (FIG. 13D). These studies indicate that Ad.mda-7 displays similar cancer-specific growth-suppressive and apoptosis-inducing properties in TRAMP-C2 prostate cancer cells, with no toxic effects in normal cells.

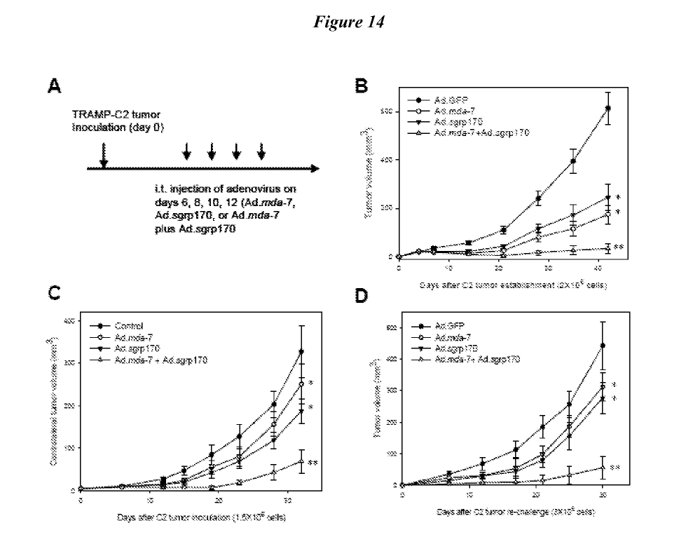

Intratumoral delivery of adenovirus encoding secretable grp170 enhances therapeutic efficacy of mda-7/IL-24-based gene therapy. We next assessed the therapeutic effects of intratumoral (i.t) injection of apoptosis-inducing mda-7/IL-24 in conjunction with immunostimulatory adjuvant grp170 on weakly immunogenic TRAMP-C2 tumors (FIG. 14). Immunization with irradiated C2 cells did not protect mice from subsequent tumor challenge (data not shown). Groups of C57BL/6 mice were established in the right flank with C2 tumors. When tumor size reached 5-mm in diameter, mice were treated with Ad.GFP, Ad.mda-7, Ad.sgrp170 or Ad.mda-7 plus Ad.sgrp170 (total 5×10⁸ pfu per injection) (FIG. 14A). It was observed that administration of Ad.GFP or PBS (data not shown) had little effect on C2 tumors, whereas treatment with either Ad.mda-7 or Ad.sgrp170 significantly delayed tumor growth. However, treatment with Ad.mda-7 combined with Ad.sgrp170 exhibited much more potent tumor-suppressive activities (FIG. 14B). Additionally, tumors in 20% of mice treated with Ad.mda-7 plus Ad.sgrp170 showed complete and prolonged regression.

We additionally tested whether injection with Ad.mda-7 and Ad.sgrp170 could induce systemic immune responses that can control established tumors. The C2 tumors were inoculated into both the left and right flanks of mice. The tumor-bearing mice were treated in the right flank only with Ad.mda-7, Ad.sgrp170, Ad.mda-7 plus Ad.sgrp170 or PBS (FIG. 14C). By comparison with the control group, mice receiving Ad.mda-7 plus Ad.sgrp170 showed a significant inhibition in tumor growth on the untreated, contralateral flank, whereas in mice injected with Ad.mda-7, growth of contralateral tumors was essentially unimpeded. Although injection of Ad.sgrp170 appeared to delay tumor growth to some extent, there was no statistical significance when compared to the control group.

Nearly half of prostate cancer patients with clinically localized tumor undergo surgery to remove all or most of the cancer during the early phase of their disease. Therefore, we examined whether the combined in situ tumor therapies using Ad.mda-7 and Ad.sgrp170 prior to surgery could prevent tumor growth after rechallenge. Mice established with C2 tumors in the right flank received Ad.GFP, Ad.mda-7, Ad.sgrp170 or Ad.mda-7 plus Ad.sgrp170. All primary tumors were surgically removed one week after the last treatment. The mice were then challenged with C2 tumor cells in the left flank 10 days later. As shown in FIG. 3D, mice treated with Ad.mda-7 plus Ad.sgrp170 were protected from rechallenge with C2 tumor, whereas those treated with Ad.GFP or Ad.mda-7 alone were still susceptible. It was seen that Ad.sgrp170 treatment failed to protect mice from rechallenge with the same tumor, suggesting that the mda-7/IL-24-mediated tumor apoptosis plays an important role in induction of antitumor responses. Moreover, treatment of local tumor with Ad.mda-7 plus Ad.sgrp170 prior to surgery was observed to more effectively reduce lung metastasis established by intravenous inoculation of C2 tumor cells, as compared to the treatment with either Ad.mda-7 or Ad.sgrp170 (data not shown).

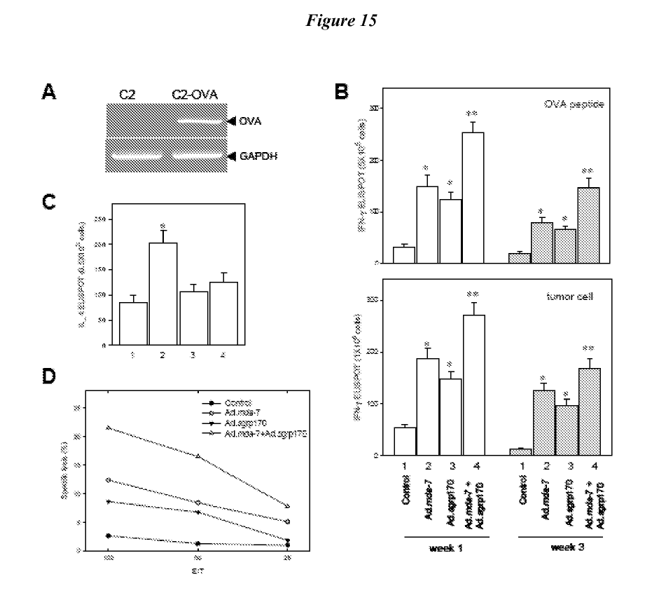

Co-administration of Ad.mda-7 and Ad.sgrp170 induces an antigen and tumor-specific immunity. To facilitate immuno-monitoring of antigen-specific immune response elicited by the combined therapies, we established TRAMP-C2 tumor cell line expressing a model antigen OVA. The expression of OVA gene in transduced C2 cells was confirmed by RT-PCR (FIG. 15A). Mice established with C2-OVA tumors were treated with Ad.mda-7, Ad.sgrp170, or Ad.mda-7 plus Ad.sgrp170. Splenocytes were isolated from the treated mice one or three weeks following the last injection. The ELISPOT assay was used to examine the OVA-specific IFN-γ production by splenocytes upon stimulation with MHC I-restricted CTL epitope for OVA, i.e., OVA₂₅₇₋₂₆₄ (SIINFEKL) (FIG. 15B, top). Compared with those from Ad.mda-7 or Ad.sgrp170 treated mice, a significant elevation in the level of IFN-γ was observed in cells from animals treated with Ad.mda-7 plus Ad.sgrp170. It is also evident that splenocytes of Ad.mda-7-treated mice produced more IFN-γ compared with those from control mice, which agrees with the previous report showing the immunomodulatory effects of mda-7/IL-24 (32). However, levels of IFN-γ secreted by these cells were much lower than those derived from the group treated with combined therapies, even when examined three weeks following the treatment. Similar results were obtained when splenocytes from the treated mice were stimulated with mitomycin C-treated tumor cells (FIG. 15B, bottom), suggesting that introduction of sgrp170 into Ad.mda-7 treated tumor promotes tumor-specific IFN-γ production.

Furthermore, ELISPOT assay was performed to measure the tumor-specific secretion of IL-4 by splenocytes from the treated animals. It was observed that splenocytes of Ad.mda-7 treated mice produced higher levels of IL-4 compared to cells from animals treated with Ad.sgrp170 or Ad.mda-7 plus Ad.sgrp170 (FIG. 15C). Effector CD8′-T cell function, i.e., cytolytic activity, was assessed by chromium release assay using C2-OVA tumor cell as a target (FIG. 15D). At effector-target (E:T) ratios of 100:1 and 50:1, significantly increased cytolytic activities were observed in Ad.mda-7 plus Ad.sgrp170-treated group when compared to Ad.mda-7 or Ad.sgrp170-treated group. Similar results were obtained when OVA₂₅₇₋₂₆₄-pulsed C2 cells were used as targets in the cytolytic assays (data not shown).

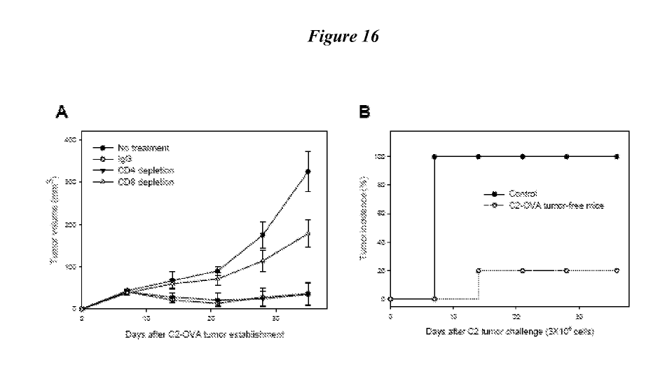

CD8⁺ T-cells are primarily involved in the systemic antitumor effects provided by the combined gene therapies. We next examined the immune effector cells involved in the antitumor immunity generated by the combined in situ therapies. C2-OVA tumor-bearing mice were depleted of CD4⁺ or CD8⁺ T-cell subsets by antibody injections prior to the initiation of treatment (FIG. 16A). It was found that depletion of CD8⁺ T-cell abrogated the therapeutic efficacy of the combined treatments, indicating that CD8⁺ T-cell plays a critical role in tumor eradiation. However, antitumor immunity remained intact in mice depleted of CD4⁺ T-cell or those treated with control IgG. In addition, a higher cure rate following administration of Ad.mda-7 and Ad.sgrp170 was observed in C2-OVA tumor model compared to parental C2 tumor model (50% versus 20%), most likely due to the surrogate antigen OVA transduced into the cells. To determine whether the antitumor immune response in C2-OVA tumor model was directed against only OVA antigen, we re-challenged C2-OVA tumor free mice which had undergone the combined treatments with parental C2 tumor (FIG. 16B). 80% of mice were resistant to the secondary tumor challenge, suggesting that the treatment of C2-OVA tumor with Ad.mda-7 and Ad.sgrp170 also induced immune responses against other endogenous antigens in addition to OVA. However, these mice developed aggressively growing tumors when re-challenged with B16 melanoma tumor (data not shown), indicating tumor specificity of the antitumor immune response.

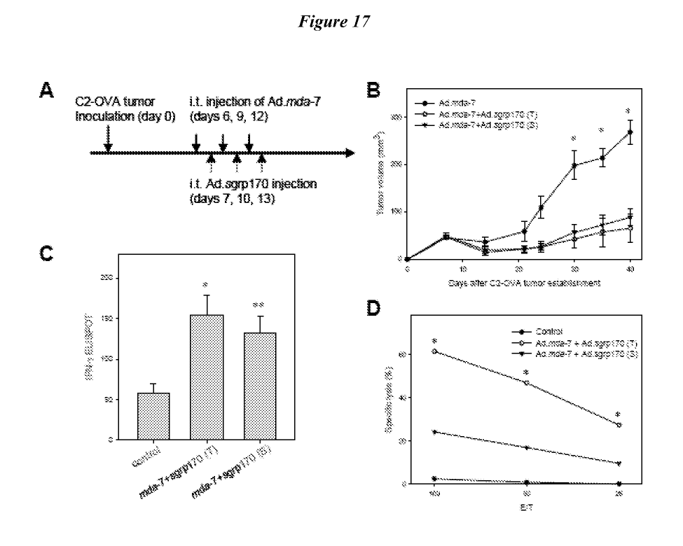

Separate intratumoral administration of Ad.mda-7 and Ad.sgrp170 is capable of inducing antitumor immunity. To determine whether i.t. injection of mda-7/IL-24 and sgrp170 at the same time is required for the generation of systemic immunity, a modified treatment protocol was used to deliver Ad.mda-7 and Ad.sgrp170 at different time points as described in FIG. 17A. In contrast to Ad.mda-7-treated group, significant enhancement of mda-7/IL-24-targeted therapy by sgrp170 was observed in mice receiving the two therapeutic agents either together (T) or separately (S) (FIG. 17B, p<0.01 for Ad.mda-7 plus Ad.sgrp170 (T) or Ad.mda-7 plus Ad.sgrp170 (S) versus Ad.mda-7 group). Although administration of Ad.mda-7 and Ad.sgrp170 together seemed to provide an improved control of treated tumors compared to the delivery of these two molecules on different days, there was no statistic difference between these two treatment groups (p>0.05). Upon stimulation with the OVA₂₅₇₋₂₆₄ peptide, splenocytes derived from C2-OVA-bearing mice treated with either with Ad.mda-7 plus Ad.sgrp170 (T) or Ad.mda-7 plus Ad.sgrp170 (S) both displayed a robust, but comparable production of IFN-γ (FIG. 17C). However, cytolytic activity assays showed that concurrent delivery of Ad.mda-7 and Ad.sgrp170 at the same time promoted a more potent CTL response than separate administration of Ad.mda-7 and Ad.sgrp170 (FIG. 17D).

Identification of mda-7/IL-24, a cancer-specific apoptosis-inducing cytokine, has provided a unique opportunity to develop molecular-targeted cancer therapies (31). However, to achieve ultimate tumor control, it would be ideal if integrating an immunotherapeutic protocol into the design of the mda-7-based treatment can generate systemic antitumor immunity. In this study we have evaluated a novel adenovirus-mediated gene therapy involving tumor apoptosis-inducing gene mda-7/IL-24 and an immunostimulatory adjuvant grp170. In an established mouse prostate cancer TRAMP-C2 model, concurrent intratumoral administration of secretable grp170 significantly enhanced therapeutic efficacy of the mda-7/IL-24-based gene therapy strategy via promoting antigen and/or tumor-specific immune responses.

Two features make grp170 a highly potent, “physiological” mammalian adjuvant that can be used for active immunotherapy: a cross-priming carrier and activator of innate immunity (25, 26). Based on our recent report demonstrating that extracellular targeting of grp170 dramatically improves the immunogenicity of poorly immunogenic tumors, including melanoma (27) and prostate cancer (Gao et al., unpublished data), we postulate that tumor-specific killing by adenovirus-mediated mda-7/IL-24 expression, taken together with simultaneous release of tumor-derived grp170, will provide both ‘danger’ signals and tumor-associated antigens to APCs (e.g., DCs), leading to strong tumor-specific immunity.

Consistent with other reports, we have shown that replication-incompetent adenoviral vector encoding mda-7/IL-24 is capable of selectively inducing tumor-specific apoptosis in mouse TRAMP-C2 tumor line in vitro whereas no harmful effects are observed in normal cells. However, intratumoral administration of either Ad.mda-7 or Ad.sgrp170 alone is not sufficient to augment a robust systemic antitumor response in this prostate cancer model, even though a significant delay in tumor growth was observed in treated C2 tumors. Our studies demonstrate that the unique combination of tumor-suppressor mda-7/IL-24 and immunochaperone grp170 resulted in a highly effective control of not only local treated tumors, but also distant untreated tumors.

A significant increase in antigen-specific CD8⁺ T-cell frequency and tumor-specific cytolytic activity were displayed in mice treated with Ad.mda-7 and Ad.sgrp170, as compared to mice treated with either Ad.mda-7 or Ad.sgrp170, suggesting that extracellular targeting of ER chaperone grp170 is a critical factor for initiation of tumor-specific and effective systemic immune responses. An early study by Miyahara et al. showed that adenoviral-mediated mda-7/IL-24 transfer induced anticancer immunity in UV-induced fibrosarcoma model (32). In agreement with the finding, we have shown that mda-7/IL-24 did exhibit immunostimulatory activities in our experiment, as indicated by enhanced IFN-γ production in OVA or tumor-specific T-cells from Ad.mda-7 treated mice. However, the treatment failed to elicit effective systemic immunity against distant or secondary TRAMP-C2 tumors, which have been known to be poorly immunogenic. The discrepancy between our data and data by Ramesh's group might be related to the use of different tumor models. Interestingly, it was observed that splenocytes from Ad.mda-7-treated mice consistently produced higher levels of IL-4 than those from animals treated with Ad.sgrp170 or Ad.mda-7 plus Ad.sgrp170 (FIG. 15C), which lends support to an early study in which murine mda-7, also called IL-4-induced secreted protein (FISP), was postulated to be a type 2 cytokine (33). However, more studies are needed to determine the pleiotropic functions of this novel tumor suppressor.

In light of the findings that stress proteins are capable of stimulating Th1-polarizing cytokine production (34-36) and activating antigen-specific CD8 T-cells (37, 38), it is tempting to postulate that, in addition to facilitating antigen transport and subsequent uptake and presentation by APCs, the secreted grp170 acts as a Th1 polarizing adjuvant during mda-7/IL-24-induced tumor cell apoptosis, promoting IFN-γ-producing Type 1 CD8⁺ T cells (Tc1). It has been shown that Tc1 cells are more effective in delaying tumor growth and progression than that of functionally distinct Tc2 cells (39). Studies to dissect the molecular mechanisms underlying the combined therapies are currently underway. Nonetheless, it is evident that the presence of the secretable form of grp170 in tumor microenvironment markedly increases the immunogenicity of the mda-7/IL-24-mediated tumor cell death and improves the therapeutic efficacy of mda-7/IL-24-based tumor-targeted therapy. In addition, the results obtained from our studies have also eliminated the concerns that intratumoral injection of grp170 might antagonize the pro-apoptotic activity of mda-7/IL-24, since stress protein generally plays a protective role in various cellular processes, including apoptosis. However, co-infection of TRAMP-C2 cells with sgrp170 has little impact on Ad.mda-7-mediated tumor apoptosis and growth inhibition in vitro. It may be due to the fact that there was no accumulation of the secreted form of grp170 in the ER and extracellular targeting of grp170 does not affect the expression of other ER chaperones, e.g., grp78/Bip (27), which has been implicated as an intracellular target for mda-7/IL-24 (13). Furthermore, evaluation of the combined strategies targeting both tumor and immune compartments in vivo indicates that both tumor-specific apoptosis induction by mda-7/IL-24 and immunostimulatory adjuvant activities of sgrp170 contribute to the synergistic or additive antitumor effects provided by the combined therapies.

In support of our results obtained from ELISPOT and CTL assays, antibody depletion studies in vivo further confirmed that CD8⁺-T cell is required for the enhanced antitumor response observed in mice treated with Ad.mda-7 and Ad.sgrp170. Given that depletion of CD8⁺ T cells does not completely diminish the antitumor effects mediated by the combined therapies, it is possible that other immune effector cells may participate in the tumor control, such as NK cells. Our previous study showed that both NK cell and CD8⁺-T cells are required for tumor rejection elicited by vaccination with grp170-secreting tumor cells (27). In addition, studies from other groups also reported that extracellular targeting of stress protein or intratumoral administration of stress protein (e.g., hsp70)-encoding adenovirus promotes expansion and activity of NK cell (40, 41).

The enhanced antigen-specific CTL response and systemic antitumor immunity in animals treated with the Ad.sgrp170 in conjunction with Ad.mda-7 strongly indicates that extracellular targeting of grp170 in the tumor milieu promotes antigen priming and cellular immunity. Our earlier studies have shown that the largest ER chaperone grp170 is highly efficient in binding polypeptide chains (20, 27) and grp170 purified from tumor exhibits a more potent therapeutic efficacy than other stress proteins (23). The enhanced immunogenicity may be attributed to its highly efficient chaperoning capability. Structure deletion studies revealed that grp170 contains two unique substrate-binding regions, i.e., the β-sheet domain and the C-terminal helix domain (20). Furthermore, our recent studies have demonstrated that molecular chaperoning function is essential for the high potency of grp170 as an immune adjuvant, e.g., interaction with APCs, antigen binding, and generation of antitumor immunity (25). Supporting evidence also came from other groups suggesting that the ability of chaperone-peptide complexes to generate an antigen-specific CTL response correlates with the affinity with which the chaperone binds substrates or peptides (42, 43). Given the high potency of grp170 as an antigen carrier and immunostimulatory adjuvant compared to other stress proteins, we are currently evaluating therapeutic strategies combining Ad.sgrp170 with tumor-targeted standard cancer treatments, e.g., radiotherapy and chemotherapy in preclinical models.

This gene-based vaccine strategy tested here has several important advantages for clinical application. It has unique capacity to induce individual tumor-specific immune responses against a broad array of mutated tumor antigens, obviating the need to prepare vaccines from surgically resected tumor specimens ex vivo. Furthermore, the intratumoral immunotherapy drastically reduces the possibility of tumor escape due to antigen loss or tumor heterogeneity, since the approach uses the tumor against itself and grp170 derived from tumor cells is directed against a diverse antigenic repertoire. In contrast to conventional stress protein-based vaccination approach, the efficacy of which is strictly limited by the quantity of stress proteins and the availability of tumor specimens, the mda-7/IL-24 and sgrp170-based therapy described here is universally applicable and more cost effective, since the vaccine is generated at the site of the patient's own tumor using his own tumor antigens. Compared to other tumor-targeted gene therapy approaches for inducing cell death in rapidly dividing cells, e.g., replication-competent oncolytic adenovirus (44) and a herpes simplex virus thymidine kinase suicide gene (HSV-TK) (41), mda-7/IL-24-based approach should display enhanced safety in the clinic because of the cancer specificity of mda-7/IL-24 and its ‘bystander’ activities (10, 31).

Among the solid tumors, CaP is ideally suited for the first test of efficacy of this idea because this non-essential organ expresses a wide array of unique antigens and highly accessible to gene transfer by using digital or transrectal ultrasound guidance (45). In addition, primary CaP is relatively slow growing and thus sequential gene therapy approaches can be incorporated safely into treatment strategies. Serum prostate-specific antigen (PSA) can be easily used to monitor treatment response. The strategy may also prove effective against CaP cells that are androgen-independent, since mda-7/IL-24 causes release of tumor antigens from CaP regardless of their androgen sensitivity. Thus, this approach may be useful in combination with androgen-deprivation therapy and can be tested in hormone-refractory CaP.

While surgery is necessary for cure, it is possible that early removal of localized prostate cancer by radical prostatectomy may preclude an opportunity to generate effective long-term immune protection. We found that surgical removal of TRAMP-C2 tumor alone does not elicit tumor-protective immunity in mice (data not shown). In this example, we demonstrate that antitumor immunity generated by the combined therapies still remains intact in animals following surgical removal of the treated local tumors. Therefore, one possibility would be to treat patients with clinically localized prostate cancer but high risk for recurrence with the molecular-targeted therapies described here before surgical removal of the primary tumor. The primed individual tumor-specific immune responses could provide improved protection against CaP recurrence or metastasis. Furthermore, our studies have also shown that a comparable antitumor response can be generated in mice treated with Ad.mda-7 and Ad.sgrp170 either together or separately. However, it is evident that administration of these therapeutic agents at the same time elicited a more robust CTL response than delivery of the two molecules at different times, as indicated by the higher level of cytolytic activity in effector T-cells. It appears that simultaneous introduction of mda-7/IL-24 and sgrp170 is beneficial in the clinic setting; however, additional experiments should be carried out to further define and optimize the treatment protocol.

Taken together, this study evaluated a dual molecular target-based therapy with significant promise for improving prostate cancer therapy, which may also have potential applications for other neoplastic diseases. This novel approach exploits an immunostimulatory chaperone molecule, i.e., grp170, in combination with a nontoxic cancer-specific apoptosis-inducing gene, mda-7/IL-24. Given the encouraging phase I clinical studies with Ad.mda-7 (46-48) and minimal toxic side effects in mouse models and clinical trials with stress protein-based vaccines (49), this strategy merits further evaluation for potential clinical use. Moreover, these data provide a rationale for combining chaperone grp170 with other conventional therapeutic modalities (e.g., radiotherapy) to induce durable systemic immunity, which may provide an even greater opportunity for prostate cancer patients to be cured of their cancer.

### REFERENCES

1. Jiang H, Lin J J, Su Z Z, Goldstein N I, Fisher P B. Subtraction hybridization identifies a novel melanoma differentiation associated gene, mda-7, modulated during human melanoma differentiation, growth and progression. Oncogene 1995; 11:2477-86.
2. Jiang H, Su Z Z, Lin J J, Goldstein N I, Young C S, Fisher P B. The melanoma differentiation associated gene mda-7 suppresses cancer cell growth. Proc Natl Acad Sci U S A 1996; 93:9160-5.
3. Su Z Z, Madireddi M T, Lin J J, et al. The cancer growth suppressor gene mda-7 selectively induces apoptosis in human breast cancer cells and inhibits tumor growth in nude mice. Proc Natl Acad Sci USA 1998; 95:14400-5.
4. Saeki T, Mhashilkar A, Chada S, Branch C, Roth J A, Ramesh R. Tumor-suppressive effects by adenovirus-mediated mda-7 gene transfer in non-small cell lung cancer cell in vitro. Gene Ther 2000; 7:2051-7.
5. Fisher P B, Gopalkrishnan R V, Chada S, et al. mda-7/IL-24, a novel cancer selective apoptosis inducing cytokine gene: from the laboratory into the clinic. Cancer Biol Ther 2003; 2:S23-37.
6. Lebedeva I V, Su Z Z, Chang Y, Kitada S, Reed J C, Fisher P B. The cancer growth suppressing gene mda-7 induces apoptosis selectively in human melanoma cells. Oncogene 2002; 21:708-18.
7. Ramesh R, Mhashilkar A M, Tanaka F, et al. Melanoma differentiation-associated gene 7/interleukin (IL)-24 is a novel ligand that regulates angiogenesis via the IL-22 receptor. Cancer Res 2003; 63:5105-13.
8. Su Z Z, Lebedeva I V, Sarkar D, et al. Melanoma differentiation associated gene-7, mda-7/IL-24, selectively induces growth suppression, apoptosis and radiosensitization in malignant gliomas in a p53-independent manner. Oncogene 2003; 22:1164-80.
9. Yacoub A, Mitchell C, Lebedeva I V, et al. mda-7 (IL-24) Inhibits growth and enhances radiosensitivity of glioma cells in vitro via JNK signaling. Cancer Biol Ther 2003; 2:347-53.
10. Su Z, Emdad L, Sauane M, et al. Unique aspects of mda-7/IL-24 antitumor bystander activity: establishing a role for secretion of MDA-7/IL-24 protein by normal cells. Oncogene 2005; 24:7552-66.
11. Sarkar D, Su Z Z, Lebedeva I V, et al. mda-7 (IL-24) Mediates selective apoptosis in human melanoma cells by inducing the coordinated overexpression of the GADD family of genes by means of p38 MAPK. Proc Natl Acad Sci USA 2002; 99:10054-9.
12. Mhashilkar A M, Stewart A L, Sieger K, et al. MDA-7 negatively regulates the beta-catenin and PI3K signaling pathways in breast and lung tumor cells. Mol Ther 2003; 8:207-19.
13. Gupta P, Walter M R, Su Z Z, et al. BiP/GRP78 is an intracellular target for MDA-7/IL-24 induction of cancer-specific apoptosis. Cancer Res 2006; 66:8182-91.
14. Sanda M G, Restifo N P, Walsh J C, et al. Molecular characterization of defective antigen processing in human prostate cancer. J Natl Cancer Inst 1995; 87:280-5.
15. Savill J, Fadok V. Corpse clearance defines the meaning of cell death. Nature 2000; 407:784-8.
16. Steinman R M, Turley S, Mellman I, Inaba K. The induction of tolerance by dendritic cells that have captured apoptotic cells. J Exp Med 2000; 191:411-6.
17. Melcher A, Todryk S, Hardwick N, Ford M, Jacobson M, Vile R G. Tumor immunogenicity is determined by the mechanism of cell death via induction of heat shock protein expression. Nat Med 1998; 4:581-7.
18. Todryk S M, Melcher A A, Dalgleish A G, Vile R G. Heat shock proteins refine the danger theory. Immunology 2000; 99:334-7.
19. Restifo N P. Building better vaccines: how apoptotic cell death can induce inflammation and activate innate and adaptive immunity. Curr Opin Immunol 2000; 12:597-603.
20. Park J, Easton D P, Chen X, MacDonald I J, Wang X Y, Subjeck J R. The chaperoning properties of mouse grp170, a member of the third family of hsp70 related proteins. Biochemistry 2003; 42:14893-902.
21. Dierks T, Volkmer J, Schlenstedt G, et al. A microsomal ATP-binding protein involved in efficient protein transport into the mammalian endoplasmic reticulum. Embo J 1996; 15:6931-42.
22. Spee P, Subjeck J, Neefjes J. Identification of novel peptide binding proteins in the endoplasmic reticulum: ERp72, calnexin, and grp170. Biochemistry 1999; 38:10559-66.
23. Wang X Y, Kazim L, Repasky E A, Subjeck J R. Characterization of heat shock protein 110 and glucose-regulated protein 170 as cancer vaccines and the effect of fever-range hyperthermia on vaccine activity. J Immunol 2001; 166:490-7.
24. Facciponte J G, Wang X Y, Subjeck J R. Hsp110 and Grp170, members of the Hsp70 superfamily, bind to scavenger receptor-A and scavenger receptor expressed by endothelial cells-I. Eur J Immunol 2007; 37:2268-79.
25. Park J, Facciponte J G, Chen X, et al. Chaperoning function of stress protein grp170, a member of the hsp70 superfamily, is responsible for its immunoadjuvant activity. Cancer Res 2006; 66:1161-8.
26. Manjili M H, Park J, Facciponte J G, Wang X-Y, Subjeck J R. Immunoadjuvant chaperone, GRP170, induces “danger signals” upon interaction with dendritic cells. Immunol Cell Biol 2006.
27. Wang X Y, Arnouk H, Chen X, Kazim L, Repasky E A, Subjeck J R. Extracellular targeting of endoplasmic reticulum chaperone glucose-regulated protein 170 enhances tumor immunity to a poorly immunogenic melanoma. J Immunol 2006; 177:1543-51.
28. Foster B A, Gingrich J R, Kwon E D, Madias C, Greenberg N M. Characterization of prostatic epithelial cell lines derived from transgenic adenocarcinoma of the mouse prostate (TRAMP) model. Cancer Res 1997; 57:3325-30.
29. Lebedeva I V, Sarkar D, Su Z Z, et al. Bcl-2 and Bcl-x(L) differentially protect human prostate cancer cells from induction of apoptosis by melanoma differentiation associated gene-7, mda-7/IL-24. Oncogene 2003; 22:8758-73.
30. Wang X Y, Chen X, Manjili M H, Repasky E, Henderson R, Subjeck J R. Targeted immunotherapy using reconstituted chaperone complexes of heat shock protein 110 and melanoma-associated antigen gp100. Cancer Res 2003; 63:2553-60.
31. Fisher P B. Is mda-7/IL-24 a “magic bullet” for cancer? Cancer Res 2005; 65:10128-38.
32. Miyahara R, Banerjee S, Kawano K, et al. Melanoma differentiation-associated gene-7 (mda-7)/interleukin (IL)-24 induces anticancer immunity in a syngeneic murine model. Cancer Gene Ther 2006; 13:753-61.
33. Schaefer G, Venkataraman C, Schindler U. Cutting edge: FISP (IL-4-induced secreted protein), a novel cytokine-like molecule secreted by Th2 cells. Journal of Immunology 2001; 166:5859-63.
34. Wang Y, Kelly C G, Singh M, et al. Stimulation of Th1-polarizing cytokines, C—C chemokines, maturation of dendritic cells, and adjuvant function by the peptide binding fragment of heat shock protein 70. J Immunol 2002; 169:2422-9.
35. Baker-LePain J C, Sarzotti M, Nicchitta C V. Glucose-regulated protein 94/glycoprotein 96 elicits bystander activation of CD4+ T cell Th1 cytokine production in vivo. J Immunol 2004; 172:4195-203.
36. Wan T, Zhou X, Chen G, et al. Novel heat shock protein Hsp70L1 activates dendritic cells and acts as a Th1 polarizing adjuvant. Blood 2004; 103:1747-54.
37. Chen C H, Wang T L, Hung C F, et al. Enhancement of DNA vaccine potency by linkage of antigen gene to an HSP70 gene. Cancer Res 2000; 60:1035-42.
38. Ramirez S R, Singh-Jasuja H, Warger T, et al. Glycoprotein 96-activated dendritic cells induce a CD8-biased T cell response. Cell Stress Chaperones 2005; 10:221-9.
39. Dobrzanski M J, Reome J B, Hylind J C, Rewers-Felkins K A. CD8-mediated type 1 antitumor responses selectively modulate endogenous differentiated and nondifferentiated T cell localization, activation, and function in progressive breast cancer. J Immunol 2006; 177:8191-201.
40. Strbo N, Oizumi S, Sotosek-Tokmadzic V, Podack E R. Perforin is required for innate and adaptive immunity induced by heat shock protein gp96. Immunity 2003; 18:381-90.
41. Ren W, Strube R, Zhang X, Chen S Y, Huang X F. Potent tumor-specific immunity induced by an in vivo heat shock protein-suicide gene-based tumor vaccine. Cancer Res 2004; 64:6645-51.
42. Moroi Y, Mayhew M, Trcka J, et al. Induction of cellular immunity by immunization with novel hybrid peptides complexed to heat shock protein 70. Proc Natl Acad Sci USA 2000; 97:3485-90.
43. MacAry P A, Javid B, Floto R A, et al. HSP70 peptide binding mutants separate antigen delivery from dendritic cell stimulation. Immunity 2004; 20:95-106.
44. Huang X F, Ren W, Rollins L, et al. A broadly applicable, personalized heat shock protein-mediated oncolytic tumor vaccine. Cancer Res 2003; 63:7321-9.
45. Ayala G, Satoh T, Li R, et al. Biological response determinants in HSV-tk+ganciclovir gene therapy for prostate cancer. Mol Ther 2006; 13:716-28.
46. Cunningham C C, Chada S, Merritt J A, et al. Clinical and local biological effects of an intratumoral injection of mda-7 (IL24; INGN 241) in patients with advanced carcinoma: a phase I study. Mol Ther 2005; 11:149-59.
47. Tong A W, Nemunaitis J, Su D, et al. Intratumoral injection of INGN 241, a nonreplicating adenovector expressing the melanoma-differentiation associated gene-7 (mda-7/IL24): biologic outcome in advanced cancer patients. Mol Ther 2005; 11:160-72.
48. Fisher P B, Sarkar D, Lebedeva I V, et al. Melanoma differentiation associated gene-7/interleukin-24 (mda-7/IL-24): novel gene therapeutic for metastatic melanoma. Toxicol Appl Pharmacol 2007; 224; 300-7.
49. Wang X Y, Facciponte J G, Subjeck J R. Molecular chaperones and cancer immunotherapy. Handb Exp Pharmacol 2006:305-29.

### Example 3

This Example describes systemic delivery of the virus in therapy resistant Bcl-X_{L} overexpressing tumor xenografts in nude mice using UTMD.

Systemic delivery of the antitumor gene mda-7/IL-24 by microbubbles was studied. For this proof-of-principle study, we used DU-145-Bcl-x_{L} (DU-145 ectopically express Bcl-X_{L}), an Ad.mda-7-resistant variant of DU-145 human prostate carcinoma cells. DU-Bcl-x_{L} tumor xenografts were established on both flanks of nude mice by injecting 2×10⁶ cells in each side of the animal. Treatment was initiated when the tumor reached a size of 250-350 mm³. Four injections of the various Ad/microbubble complexes into the tail vein once per week (total of 4 weeks) were administered followed by ultrasound (US) for 10 min on the tumor on the right side only. No treatment was performed on the tumor xenografted on the left flank. Animals receiving the Ad-GFP-microbubble complexes plus US treatment showed no statistically significant effect on the growth of DU-145-Bcl-x_{L} tumors. Microbubbles (Ad.PEG-E1A-mda-7) elicited a sustained growth inhibition of the therapy resistant DU-Bc1-x_{L} tumor xenografts in both primary and distant tumors. Western blot analysis of total protein extracts from the harvested tumors showed expression of MDA-7/IL-24 protein in both the tumor samples implanted on the right and left flank validating the “bystander” effects of MDA-7/IL-24. The amplified expression of MDA-7/IL-24 in the non-injected left tumor may also reflect secondary viral infection by the CRAd. Control tumors treated with Ad-GFP-microbubble complexes were mostly TUNEL negative. Treatments with the microbubbles plus US disrupted the tumor cyto-architecture, which correlated with an increase in the number of TUNEL positive tumor cells in both sonoporated right and untreated left tumors. Control tumors treated with Ad-GFP-microbubble complexes were TUNEL negative. B-mode ultrasound-scan of DU-Bcl-x_{L} tumors showed dramatic volume reductions in the tumors after 2 and 4 wks of treatments with microbubble complexes and US leading to the eradication of the tumor xenograft (data not shown). Additionally, no tumor regrowth in the primary or distant sites was evident in microbubble complex and US-treated DU-Bcl-x_{L} animals after an additional three weeks post-treatment.

### Example 4

A major challenge for effective gene therapy using Ads or recombinant proteins is the ability to specifically deliver the therapeutic directly into diseased tissue without exposure to the immune system, particularly with a systemic approach in an immune competent animal model. Progress in gene therapy has been hampered by concerns over the safety and practicality of viral vectors, particularly for intravenous delivery, and the inefficiency of currently available non-viral transfection techniques, we have developed a novel targeted-delivery approach that includes ultrasound (US) contrast agents (microbubbles) to deliver effective therapeutic reagents Ad.mda-7 or GST-MDA-7 (melanoma differentiation associated gene-7/interleukin-24), followed by ultrasound-targeted microbubble destruction (UTMD) to develop a prostate-specific therapy in a prostate cancer immune competent transgenic mouse model, Hi-Myc. We hypothesize that ultrasound (US) will increase the specificity of Ads and GST-MDA-7 delivery and limit immune clearance of the therapeutic virus and non-specific purified protein delivery by exploiting the exclusive physical properties of microbubbles enabling US-guided focused-release of entrapped materials as well as the creation of small shock waves increasing cellular permeability. In the case of Ad delivery, preliminary studies indicate that complement can achieve this objective and can inactivate not only free Ads, but also Ads adsorbed on the surface of reconstituted US contrast agents, ablating the immune response.

Protocol for systemic delivery of Ad.mda-7 into prostate of immunocompetent prostate cancer mouse model (Hi-Myc):

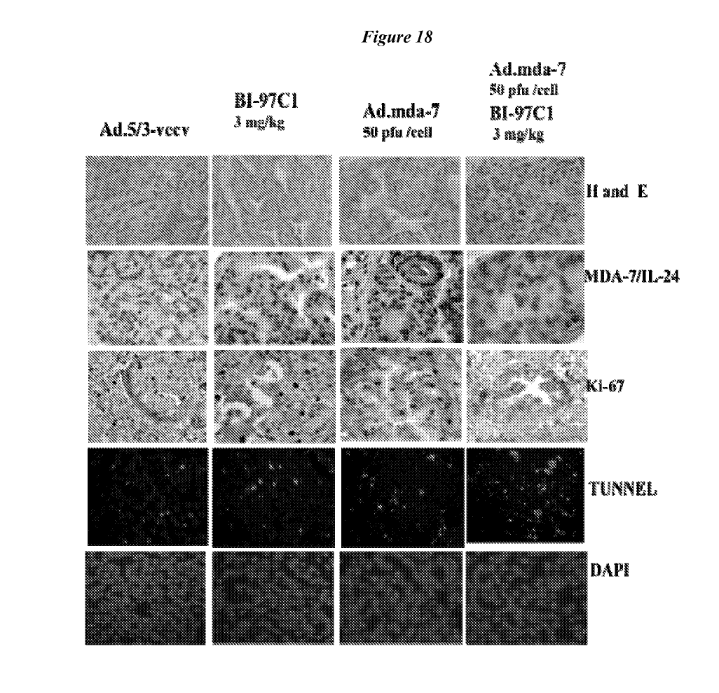

Preparation of microbubbles (MBs), US platform and UTMD. Targeson (Targeson) custom synthesis US contrast agent (perfluorocarbon MBs, encapsulated by a lipid monolayer and poly(ethyleneglycol) stabilizer) were prepared following manufacturer's instructions. MBs were reconstituted in the presence or absence of 1 ml of 0.5×10¹² plaque-forming units of Ad.vec or Ad.mda-7 and complement was achieved by incubating MBs/Ad complex with Human complement purchased from Sigma. For in vivo experiments US exposure was achieved with a Micro-Maxx SonoSite (SonoSite) US machine equipped with the transducer L25 set at 0.7 Mechanical Index, 1.8 MPa for 10 minutes. Mice were sedated in an IMPAQ6 anesthesia apparatus (VetEquip, Pleasanton, Calif.) that was saturated with 3-5% isofluorane and 10-15% oxygen with the aid of a precision. For microbubble/Ad injection (Ad.vec or Ad.mda-7) the mice were placed on a warmed mat with 37° C. circulating water for the entire procedure. A 27-gauge needle with a heparin lock was placed within a lateral tail vein for administration of contrast material. The mice received injections of 100 μl of MBs with Ads through the tail vein twice a week for 4 weeks. The mice were divided into three groups I) Ad.5/3-vec, II) Ad.5/3-mda-7, III) BI-97C1 (a novel Mc1-1 inhibitor), (IV) Ad-5/3-mda-7+BI-97C1 group. US was performed with a SonoSite scanner (SonoSite) equipped with the transducer L25 set at 0.7 Mechanical Index, 1.8 MPa for 10 minutes in the ventral side of mice in the prostatic area. At the end of the experiment, the Hi-Myc mice were sacrificed and the prostate was dissected and weighed. The harvested prostate were preserved in neutral buffered formalin at 4° C. before embedding in paraffin for immunohistochemical analysis. Our data supports efficient, targeted transgene delivery of mda-7/IL-24 in the prostate of Hi-Myc mice using UTMD (FIG. 18).

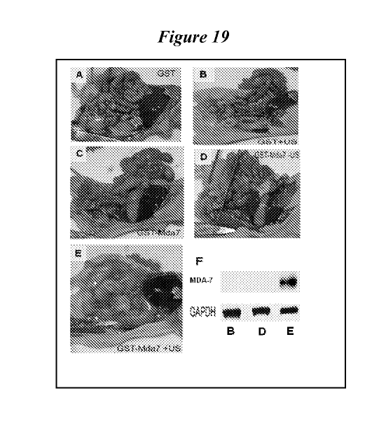

Proof-of-principle of delivering recombinant proteins using the ultrasound-targeted microbubble destruction (UTMD) approach: Although the technology to deliver nucleic acids, including viral vectors, by microbubbles is well-established, approaches for delivering bioactive proteins are a work in progress. However, several studies have shown effective administration of recombinant proteins, such as VEGF or FGF2, using phospholipid-based microbubbles or perfluorocarbon-based microbubbles enveloped in an albumin shell (either dextrose albumin or BSA). Our proof-of-principle studies reveal that GST-MDA-7 could be efficiently delivered to target tumor cells in vivo in nude mice xenograft models using UTMD, where human pancreatic cancer cells (AsPC-1) were intraperitoneally (i.p.) injected into athymic nude mice. Microbubbles were reconstituted in the presence of GST or GST-MDA-7 protein (10 nM, a dose that shows a profound cell killing effect in vitro). Four injections of the microbubble/GST or microbubble/GST-MDA-7 complexes by i.p. once a week (total of four weeks) with application of US for 10 min. The mice were sacrificed 4 weeks after treatment and tumor progression was observed followed by dissection of animals (FIG. 19). The GST, GST/microbubble with US, GST-MDA-7 and GST-MDA-7/microbubble without US groups did not induce any tumor regression (FIG. 19A, 19B, 19C, 19D), whereas the GST-MDA-7/microbubble with US group showed profound tumor regression (FIG. 19E). Moreover, we were able to detect GST-MDA-7 protein in tumor tissue only from the GST-MDA-7/microbubble group when treated with US (FIG. 19F).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of targeting a virus to cells in an animal, the method comprising:
administering a selectively replicating virus to the animal, wherein the virus is encompassed in a suspension of microbubbles, wherein the surface of the suspension does not include any virus; and
disrupting the microbubbles administered to the animal in a specific location of the animal, wherein the virus selectively replicates in the cells at said specific location.

2. The method of claim 1, wherein the animal is a human.

3. The method of claim 1 wherein the cells at said specific location are cancer cells, and wherein the cancer cells are killed as a result of replication or gene expression of the virus in the cancer cells.

4. The method of claim 3, wherein the virus expresses a chaperone protein, wherein the chaperon protein presents killed cancer cell antigens to the animal immune system.

5. The method of claim 4, wherein the chaperone protein is or includes Grp 170.

6. The method of claim 1, wherein the virus comprises a cancer-specific promoter operably linked to at least one viral gene necessary for viral replication.

7. The method of claim 6, wherein the promoter is human PEG-3 promoter.

8. The method of claim 1 wherein the virus expresses a heterologous polynucleotide, wherein the heterologous polynucleotide encodes mda-7.

9.-11. (canceled)

12. A suspension of microbubbles, the suspension comprising a selectively replicating virus wherein the virus is encompassed in a suspension of microbubbles and the surface of the suspension does not include any virus.

13. The suspension of claim 12, wherein the virus selectively replicates in cancer cells.

14.-15. (canceled)

16. The suspension of claim 12, wherein the virus comprises a cancer specific promoter operably linked to at least one viral gene necessary for viral replication.

17.-22. (canceled)

23. The method of claim 1 wherein said virus is a recombinant adenovirus.

24. The method of claim 1 wherein said cells are cancer cells, and said specific location is a cancerous site.

25. A composition for the treatment of a neoplastic disease, comprising:
a first virus encoded with a gene for a secretable immunostimulatory chaperone molecule, and
a second virus encoded with a gene for a secretable cancer specific apoptosis inducing cytokine.

26. The composition of claim 25 wherein said secretable immunostimulatory chaperone molecule is or includes Grp170.

27. The composition of claim 25 wherein said secretable cancer specific appoptosis inducing citokine is derived from a melanoma differentiated associated gene.

28. The composition of claim 25 wherein said secretable cancer specific apopttosis inducing cytokine is mda-7/IL24.

29. The composition of claim 25 wherein said first and second viruses are adenoviruses.

30. The method of claim 1 wherein said disrupting step is performed by ultrasound.


SEQUENCE LISTING (3 sequences)

SEQ ID NO: 1 - DNA, 1937 bp, Artificial Sequence
  Synthetic PEG-3 promoter
tggctgggcaacacgggttcagcccaggtttcatagtaagttccagacactcctggaaaaacaatacaggtccctgacaaaagaaaaaacaaaacaaaggaaacagaaacatgcgtttttaaaaaagaaggaggagactccatgaaggcaggccttgggtggggtcactgcttctctgtacacaggaggagaattgccaagatcttccggacagtgtggactatactgtaagaccctctcaatacagacagactggacaggcatagtgacacatcctttaatgcctgcagtactcaggaggaggtggcaggtggaacggctgttctttgaggttcaagaccagcgtggactacagagtgagttccaggacaggcagggctacacagaaaaatcctgtctgaaaacaaaacaaaacccagacagacacaccaaaaacagccaagggaccagagagatgggtcagggcctaatcacttgctactctttgcagaggacccaaatttagttcctataaccctccatgagaagcttcacaattgtctctaactcaattccacccgtgttccgacctcccatatgcaccagacatgttatactcacacatacgcacaaacacacacacacacacacacacacacacacacacacacacacacacacggaaaacatataaaataaagatttaaaaaatctttttcttttggccggggtgtgtgggagagcatctgagccatctcaccagcccagggtgcacgtctttttctttttttcggagctggggaccgaacccagagccttgtgcttgctaggcaagtgctctaccactgagctaaatccccaaccccggagcacgtctttaatcccagaatcaggaggtagaggtaatgagatcccagtgagcccaaggtcagccgagtctacaaagtgagttccaggacagccagaactaatcttggaaaaacaaacaagggctggtgaggtggttcagtagttaagaacactggctgctcttccagaggtcctgagttcattctcagtaaccacatggtggggatctgatgcctgttctgcatgcagatatacatgcagatagtgcactcctacatttaaaaaaaaaagacataaataatattttaaaacattgggcgttttgtcttctaataaaacttcactgctatcttctaataaaaattcactgctagccgcggggtgtggtgcccccatacctttaatcccaacaacttgagaggcagaggcaggcggacctttgagtttgaagctagcctggtctacagagtgagttcaagatagccacggatagtcagaaagtcctgtttcgaacctctccccaaccaaatcactcctgtaatcccagcactctggaggcagtagcaggttagtccctgcttctcagagagaggagagagagagagagagagaggagacacacacacacagagacagagaggaagagaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 2 - protein, 10 aa, Artificial Sequence
  Synthetic peptide
CDCRGRDCFC

SEQ ID NO: 3 - protein, 17 aa, Artificial Sequence
  Synthetic peptide
GSGSGSGSGSKKKKKKK